(19) United States
(12) Patent Application Publication
(10) Pub. No.: US 2010/0239551 A1
(43) Pub. Date: Sep. 23, 2010

(54) COMPOUNDS AND COMPOSITIONS AS CHANNEL ACTIVATING PROTEASE INHIBITORS

(75) Inventors: David C. Tully, San Diego, CA (US); Arnab K. Chatterjee, San Diego, CA (US); Agnes Vidal, Spring Valley, CA (US); Badry Bursulaya, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 12/525,991

(22) PCT Filed: Jan. 4, 2008
(86) PCT No.: PCT/US08/50289

§ 371 Date: Apr. 27, 2010

Related U.S. Application Data
(60) Provisional application No. 60/889,008, filed on Feb. 9, 2007.

(30) Foreign Application Priority Data
Jan. 4, 2008 (US) PCT/US2008/050289

(51) Int. Cl. A61K 31/437; C07D 227/04; A61K 31/454; C07D 401/14; A61K 31/4545; C07D 471/04; C07D 263/58; A61K 31/423; A61K 38/43; A61P 11/00; A61P 35/00; A61P 29/00; A61P 37/08; C12N 5/071

(52) U.S. Cl. 424/94.1; 546/198; 514/321; 546/187; 514/316; 546/115; 514/302; 548/217; 514/375; 435/375

(57) ABSTRACT

The invention provides compounds and pharmaceutical compositions thereof, which are useful for modulating chan-nel activating proteases, and methods for using such compounds to treat, ameliorate or prevent a condition associated with a channel activating protease, including but not limited to prostasin, PRSS22, TMPRSS11 (e.g., TMPRSS11B, TMPRSSI1E), TMPRSS2, TMPRSS3, TMPRSS4 (MTSP-2), matriptase (MTSP-1), CAP2, CAP3, trypsin, cathepsin A, or neutrophil elastase.

### CROSS-REFERENCE TO RELATED APPLICATIONS

This application claims the benefit of U.S. provisional application Ser. No. 60/889,008, filed Feb. 9, 2007, which is incorporated herein by reference in its entirety.

### TECHNICAL FIELD

The invention generally relates to channel activating protease (CAP) inhibitors.

### BACKGROUND ART

Prostasin is a trypsin-like serine protease that is present in a variety of mammalian tissues. It is a membrane anchored protease that is expressed on the extra-cellular membrane of cells but that can also be secreted into body fluids such as semen, urine and airway surface liquid. Prostasin (PRSS8), together with proteases such as matriptase, CAP2, CAP3, trypsin, PRSS22, TMPRSS11, cathepsin A, and neutrophil elastase, can stimulate the activity of the amiloride-sensitive epithelial sodium channel (ENaC). Inhibiting these enzymes can induce changes in epithelial ion transport and therefore fluid homeostasis across epithelial membranes. For example, CAP inhibition in the kidney is thought to promote diuresis, whilst CAP inhibition in the airways promotes the clearance of mucus and sputum in lung. CAP inhibition in the kidney may therefore be used therapeutically to treat hypertension. CAP inhibition in the airways prevents the stagnation of respiratory secretions that otherwise tends to make sufferers vulnerable to secondary bacterial infections.

### DISCLOSURE OF THE INVENTION

The invention provides compounds, pharmaceutical compositions and methods of using such compounds for modulating channel activating proteases (CAP). For example, the compounds and compositions of the invention may be used for modulating prostasin, PRSS22, TMPRSS11 (e.g., TMPRSS11B, TMPRSS11E), TMPRSS2, TMPRSS3, TMPRSS4 (MTSP-2), matriptase (MTSP-1), CAP2, CAP3, trypsin, cathepsin A, and neutrophil elastase.

In one aspect, the present invention provides compounds of Formula (1):

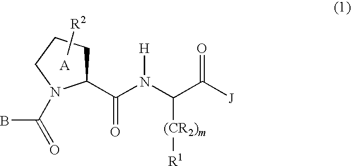

or pharmaceutically acceptable salts thereof; wherein

B is

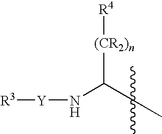

or (CR₂)ₖ—R⁵;

Y is —SO₂—, —NHCO—, —CO— or —O—C(═O)—, provided Y is SO₂ when R² is C₁₋₆ alkyl or phenyl;

J is an optionally substituted 5-12 membered monocyclic or fused heterocyclic ring comprising one or more heteroatoms selected from N, O, and S;

R¹ is H, an optionally halogenated C₁₋₆ alkyl, C₂₋₆ alkenyl or C₃₋₆ alkynyl; cyano, OH, O(CR₂)₁R⁶, SO₂R⁶, CONR(CR₂)₁R⁶, CONR⁷R⁸ or

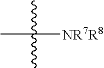

wherein R⁷ and R⁸ together with N in NR⁷R⁸ form an optionally substituted 5-7 membered heterocyclic ring attached to (CR₂)ₘ via a nitrogen atom; or R¹ is an optionally substituted C₃₋₇ cycloalkyl, aryl, or a 5-7 membered heterocyclic ring or heteroaryl having no nitrogen atoms; or

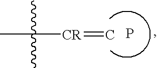

wherein ring P is an optionally substituted 5-7 membered carbocyclic ring;

R² is C₁₋₆ alkyl, C₂₋₆ alkenyl, C₂₋₆ alkynyl, aryl or -L-(CR₂)ₚ—R⁵ wherein L is O, S, S(O), SO₂ or OC(O);

R³ is C₁₋₆ alkyl, C₂₋₆ alkenyl, C₂₋₆ alkynyl or —(CR₂)₁—R⁵;

R⁴ is H, C₂₋₆ alkyl, C₂₋₆ alkenyl, —CR═CR—R⁶, C₂₋₆ alkynyl, or an optionally substituted 5-12 membered carbocyclic ring, heterocyclic ring, aryl or heteroaryl; or R⁴ is

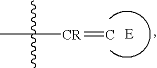

wherein ring E is an optionally substituted 5-12 membered monocyclic or fused carbocyclic or heterocyclic ring;

R⁵ and R⁶ are independently an optionally substituted 5-12 membered carbocyclic ring, heterocyclic ring, aryl or heteroaryl; or R⁶ may be C₁₋₆ alkyl or C₂₋₆ alkenyl;

each R is H, or C₁₋₆ alkyl, C₂₋₆ alkenyl, or C₂₋₆ alkynyl;

1 is 0-6; and

k, m, n, and p are independently 1-6.

In the above Formula (1), R² may be C₁₋₆ alkyl, an optionally substituted phenyl, or -L-(CR₂)ₚ—R⁵ wherein L is O.

In one embodiment, the invention provides compounds having Formula (2):

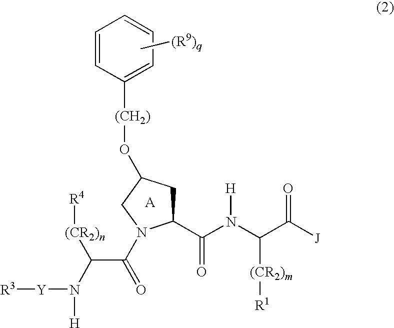

wherein J is benzoxazolyl; 1,2,3-oxadiazol-4-yl; 1,3,4-oxadiazol-2-yl; 1,2,4-oxadiazol-3-yl; oxazolo[4,5-b]pyridin-2-yl, oxazolo[4,5-c]pyridin-2-yl, oxazolo[5,4-c]pyridin-2-yl or oxazolo[5,4-b]pyridin-2-yl, each of which is optionally substituted with C₁₋₆ alkyl, halo, cyclopropyl, SO₂(C₁₋₆alkyl), OCH₃, SO₂N(CH₃)₂, SO₂NH₂, CF₃ or —(CR₂)₁—R⁵;

Y is SO₂ or —O—C(═O)—;

q is 1-5;

R⁹ is halo, C₁₋₆ alkyl, or O(C₁₋₆ alkyl); and

R, R¹, R³, R⁴, R⁵, m and n are as defined in Formula (1).

In some examples, Y in Formula (2) is SO₂ and R³ is C₁₋₆ alkyl. In other examples, R⁴ is an optionally substituted piperidinyl, cyclohexyl, phenyl,

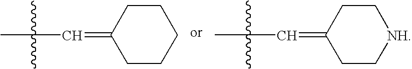

In particular examples, R⁴ is piperidinyl.

In another embodiment, the invention provides compounds having Formula (3):

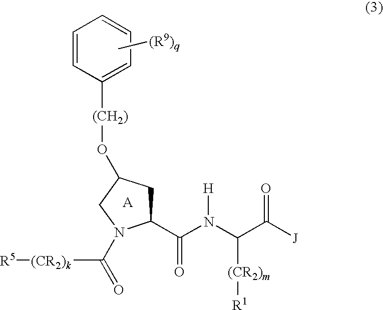

wherein R¹ is C₃₋₇ cycloalkyl or phenyl;

q is 1-5;

R⁹ is halo, C₁₋₆ alkyl, or O(C₁₋₆ alkyl); and

R, R⁵, J, k and m are as defined in Formula (1).

In the above Formula (2) and (3), q may be 1-2, and R⁹ is halo. In some examples, R⁵ in Formula (3) may be an optionally substituted cyclohexyl, piperidinyl or a thiazolyl. In particular examples, R⁵ is thiazolyl which is optionally substituted with piperidinyl.

In the above Formula (1), (2) and (3), J may be benzoxazolyl; 1,2,3-oxadiazol-4-yl; 1,3,4-oxadiazol-2-yl; 1,2,4-oxadiazol-3-yl; oxazolo[4,5-b]pyridin-2-yl, oxazolo[4,5-c]pyridin-2-yl, oxazolo[5,4-c]pyridin-2-yl or oxazolo[5,4-b]pyridin-2-yl, each of which is optionally substituted with C₁₋₆ alkyl, halo, cyclopropyl, SO₂(C₁₋₆alkyl), OCH₃, SO₂N(CH₃)₂, SO₂NH₂, CF₃ or —(CR₂)₁—R⁵. In particular examples, J is 1,2,4-oxadiazol-3-yl, which may be optionally substituted, for example, with C₁₋₆ alkyl, CF₃ or —(CR₂)₁—R⁵ wherein R⁵ is an optionally substituted phenyl or C₃₋₇ cycloalkyl.

In the above Formula (1), (2) and (3), R¹ may be C₁₋₆ alkyl, C₂₋₆ alkenyl, C₂₋₆ alkynyl, CF₃, OH, C₁₋₆ alkoxy, O(benzyl), SO₂(C₁₋₆ alkyl), CONH(C₁₋₆ alkyl), CON(C₁₋₆ alkyl)₂, or cyano; or R¹ is phenyl, cyclopropyl, cyclobutyl, cyclopentyl, cyclohexyl, tetrahydropyranyl, furanyl, piperidin-2-onyl, pyrrolidin-2-onyl, pyrrolidin-1-carbonyl,

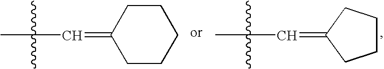

each of which is optionally substituted with halo, C₁₋₆ alkyl, C₂₋₆ alkenyl, C₃₋₆ alkynyl, cyano, OH or C₁₋₆ alkoxy.

In another aspect, the present invention provides pharmaceutical compositions comprising a compound of Formula (1), (2) or (3), and a pharmaceutically acceptable excipient.

The invention also provides methods for modulating a channel activating protease, comprising administering to a system or a mammal, a therapeutically effective amount of a compound having Formula (1), (2) or (3), or pharmaceutically acceptable salts or pharmaceutical compositions thereof, thereby modulating said channel activating protease.

In one embodiment, the invention provides a method for inhibiting a channel activating protease, comprising administering to a cell or tissue system or to a mammal, a therapeutically effective amount of a compound having Formula (1), (2) or (3) or pharmaceutically acceptable salts or pharmaceutical compositions thereof; wherein said channel activating protease is prostasin, PRSS22, TMPRSS11 (e.g., TMPRSS11B, TMPRSS11E), TMPRSS2, TMPRSS3, TMPRSS4 (MTSP-2), matriptase (MTSP-1), CAP2, CAP3, trypsin, cathepsin A, or neutrophil elastase, thereby inhibiting said channel activating protease. In particular examples, the invention provides a method for inhibiting prostasin.

In another aspect, the invention provides a method for ameliorating or treating a condition mediated by a channel activating protease, comprising administering to a cell or tissue system or to a mammal, an effective amount of a compound having Formula (1), (2) or (3), or pharmaceutically acceptable salts or pharmaceutical compositions thereof, and optionally in combination with a second therapeutic agent; wherein said channel activating protease is prostasin, PRSS22, TMPRSS11 (e.g., TMPRSS11B, TMPRSS11E), TMPRSS2, TMPRSS3, TMPRSS4 (MTSP-2), matriptase (MTSP-1), CAP2, CAP3, trypsin, cathepsin A, or neutrophil elastase, thereby treating said condition.

Furthermore, the present invention provides compounds of Formula (1), (2) or (3), for use in a method for treating a condition mediated by a channel activating protease. The present invention also provides the use of a compound of Formula (1), (2) or (3), and optionally in combination with a second therapeutic agent, in the manufacture of a medicament for treating a condition mediated by a channel activating protease.

In particular examples, the compounds of the invention may be used for treating a prostasin-mediated condition. In one embodiment, the second therapeutic agent may be an anti-inflammatory, bronchodilatory, antihistamine, anti-tussive, antibiotic or DNase, and is administered prior to, simultaneously with, or after the compound of Formula (1), (2) or (3). In some examples, the compounds of the invention are administered to bronchial epithelial cells, particularly human bronchial epithelial cells.

Examples of conditions which may be ameliorated or treated using the compounds of the invention include but are not limited to a condition associated with the movement of fluid across ion transporting epithelia or the accumulation of mucus and sputum in respiratory tissues, or a combination thereof. In some examples, the condition which may be mediated using the compounds of the invention is cystic fibrosis, primary ciliary dyskinesia, lung carcinoma, chronic bronchitis, chronic obstructive pulmonary disease, asthma or a respiratory tract infection.

Definitions

“Alkyl” refers to a moiety and as a structural element of other groups, for example halo-substituted-alkyl and alkoxy, and may be straight-chained or branched. An optionally substituted alkyl, alkenyl or alkynyl as used herein may be optionally halogenated (e.g., CF₃), or may have one or more carbons that is substituted or replaced with a heteroatom, such as NR, O or S (e.g., —OCH₂CH₂O—, alkylthiols, thioalkoxy, alkylamines, etc).

“Aryl” refers to a monocyclic or fused bicyclic aromatic ring containing carbon atoms. For example, aryl may be phenyl or naphthyl. “Arylene” means a divalent radical derived from an aryl group.

“Heteroaryl” as used herein is as defined for aryl above, where one or more of the ring members is a heteroatom. Examples of heteroaryls include but are not limited to pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, etc.

A “carbocyclic ring” as used herein refers to a saturated or partially unsaturated, monocyclic, fused bicyclic or bridged polycyclic ring containing carbon atoms, which may optionally be substituted, for example, with ═O. Examples of carbocyclic rings include but are not limited to cyclopropyl, cyclobutyl, cyclopentyl, cyclohexyl, cyclopropylene, cyclohexanone, etc.

A “heterocyclic ring” as used herein is as defined for a carbocyclic ring above, wherein one or more ring carbons is a heteroatom. For example, a heterocyclic ring may contain N, O, S, —N═, —S—, —S(O), —S(O)₂—, or —NR— wherein R may be hydrogen, C₁₋₄alkyl or a protecting group. Examples of heterocyclic rings include but are not limited to morpholino, pyrrolidinyl, pyrrolidinyl-2-one, piperazinyl, piperidinyl, piperidinylone, 1,4-dioxa-8-aza-spiro[4.5]dec-8-yl, etc.

Unless otherwise indicated, when a substituent is deemed to be “optionally substituted,” it is meant that the substituent is a group that may be substituted with one or more group(s) individually and independently selected from, for example, an optionally halogenated alkyl, alkenyl, alkynyl, alkoxy, alkylamine, alkylthio, alkynyl, amide, amino, including mono- and di-substituted amino groups, aryl, aryloxy, arylthio, carbonyl, carbocyclic, cyano, cycloalkyl, halogen, heteroalkyl, heteroalkenyl, heteroalkynyl, heteroaryl, heterocyclic, hydroxy, isocyanato, isothiocyanato, mercapto, nitro, O-carbamyl, N-carbamyl, O-thiocarbamyl, N-thiocarbamyl, C-amido, N-amido, S-sulfonamido, N-sulfonamido, C-carboxy, O-carboxy, perhaloalkyl, perfluoroalkyl, silyl, sulfonyl, thiocarbonyl, thiocyanato, trihalomethanesulfonyl, and the protected compounds thereof. The protecting groups that may form the protected compounds of the above substituents are known to those of skill in the art and may be found in references such as Greene and Wuts, Protective Groups in Organic Synthesis, 3^{rd} Ed., John Wiley & Sons, New York, N.Y., 1999, and Kocienski, Protective Groups, Thieme Verlag, New York, N.Y., 1994, which are incorporated herein by reference in their entirety.

The terms “co-administration” or “combined administration” or the like as used herein are meant to encompass administration of the selected therapeutic agents to a single patient, and are intended to include treatment regimens in which the agents are not necessarily administered by the same route of administration or at the same time.

The term “pharmaceutical combination” as used herein refers to a product obtained from mixing or combining active ingredients, and includes both fixed and non-fixed combinations of the active ingredients. The term “fixed combination” means that the active ingredients, e.g. a compound of Formula (1) and a co-agent, are both administered to a patient simultaneously in the form of a single entity or dosage. The term “non-fixed combination” means that the active ingredients, e.g. a compound of Formula (1) and a co-agent, are both administered to a patient as separate entities either simultaneously, concurrently or sequentially with no specific time limits, wherein such administration provides therapeutically effective levels of the active ingredients in the body of the patient. The latter also applies to cocktail therapy, e.g. the administration of three or more active ingredients.

The term “therapeutically effective amount” means the amount of the subject compound that will elicit a biological or medical response in a cell, tissue, organ, system, animal or human that is being sought by the researcher, veterinarian, medical doctor or other clinician.

The term “administration” and or “administering” of the subject compound should be understood to mean as providing a compound of the invention including a pro-drug of a compound of the invention to the individual in need of treatment.

As used herein, the terms “treat”, “treating” and “treatment” refer to a method of alleviating or abating a disease and/or its attendant symptoms.

The term “prostasin” may also be referred to as: human channel-activating protease (hCAP); channel-activating protease-1; and PRSS8, MERPOPS ID 501.159.

### MODES OF CARRYING OUT THE INVENTION

The invention provides compounds, pharmaceutical compositions and methods of using such compounds for modulating channel activating proteases (CAP).

In one aspect, the present invention provides compounds of Formula (1):

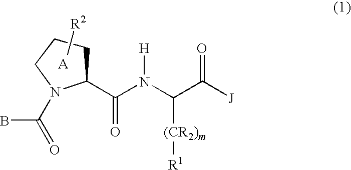

or pharmaceutically acceptable salts thereof; wherein

B is

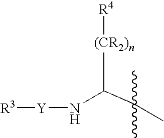

or (CR₂)ₖ—R⁵;

Y is —SO₂—, —NHCO—, —CO— or —O—C(═O)—, provided Y is SO₂ when R² is C₁₋₆ alkyl or phenyl;

J is an optionally substituted 5-12 membered monocyclic or fused heterocyclic ring comprising one or more heteroatoms selected from N, O, and S;

R¹ is H, an optionally halogenated C₁₋₆ alkyl, C₂₋₆ alkenyl or C₃₋₆ alkynyl; cyano, OH, O(CR₂)₁R⁶, SO₂R⁶, CONR(CR₂)₁R⁶, CONR⁷R⁸ or

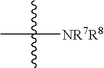

wherein R⁷ and R⁸ together with N in NR⁷R⁸ form an optionally substituted 5-7 membered heterocyclic ring attached to (CR₂)ₘ, via a nitrogen atom; or R¹ is an optionally substituted C₃₋₇ cycloalkyl, aryl, or a 5-7-membered heterocyclic ring or heteroaryl having no nitrogen atoms; or

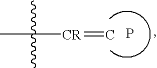

wherein ring P is an optionally substituted 5-7 membered carbocyclic ring;

R² is C₁₋₆ alkyl, C₂₋₆ alkenyl, C₂₋₆ alkynyl, aryl or -L-(CR₂)ₚ—R⁵ wherein L is O, S, S(O), SO₂ or OC(O);

R³ is C₁₋₆ alkyl, C₂₋₆ alkenyl, C₂₋₆ alkynyl or —(CR₂)₁—R⁵;

R⁴ is H, C₁₋₆ alkyl, C₂₋₆ alkenyl, —CR═CR—R⁶, C₂₋₆ alkynyl, or an optionally substituted 5-12 membered carbocyclic ring, heterocyclic ring, aryl or heteroaryl; or R⁴ is

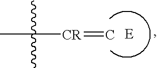

wherein ring E is an optionally substituted 5-12 membered monocyclic or fused carbocyclic or heterocyclic ring;

R⁵ and R⁶ are independently an optionally substituted 5-12 membered carbocyclic ring, heterocyclic ring, aryl or heteroaryl; or R⁶ may be C₁₋₆ alkyl or C₂₋₆ alkenyl;

each R is H, or C₁₋₆ alkyl, C₂₋₆ alkenyl, or C₂₋₆ alkynyl; 1 is 0-6; and

k, m, n, and p are independently 1-6.

In one embodiment, the invention provides compounds having Formula (2):

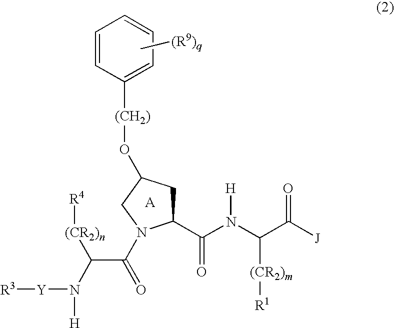

wherein J is benzoxazolyl; 1,2,3-oxadiazol-4-yl; 1,3,4-oxadiazol-2-yl; 1,2,4-oxadiazol-3-yl; oxazolo[4, 5-1)]pyridin-2-yl, oxazolo[4,5-c]pyridin-2-yl, oxazolo[5,4-c]pyridin-2-yl or oxazolo[5,4-b]pyridin-2-yl, each of which is optionally substituted with C₁₋₆ alkyl, halo, cyclopropyl, SO₂(C₁₋₆alkyl), OCH₃, SO₂N(CH₃)₂, SO₂NH₂, CF₃ or —(CR₂)₁—R⁵;

Y is SO₂ or —O—C(═O)—;

q is 1-5;

R⁹ is halo, C₁₋₆ alkyl, or O(C₁₋₆ alkyl); and

R, R¹, R³, R⁴, R⁵, m and n are as defined in Formula (1).

In another embodiment, the invention provides compounds having Formula (3):

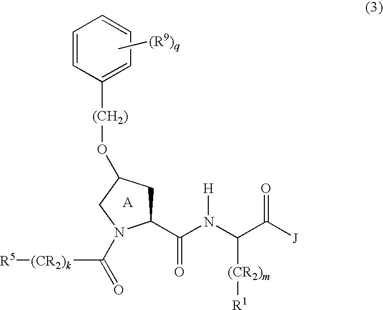

wherein R¹ is C₃₋₇ cycloalkyl or phenyl;

q is 1-5;

R⁹ is halo, C₁₋₆ alkyl, or O(C₁₋₆ alkyl); and

R, R⁵, J, k and m are as defined in Formula (1).

In other embodiments, J in the above Formula (1), (2) and (3) is selected from the group including but not limited to imidazolin-2-yl; imidazol-2-yl; oxazolin-2-yl; oxazol-2-yl; thiazolin-2-yl; thiazol-2-yl; thiazol-5-yl; 1,3,4-thiadiazol-2-yl; 1,2,4-thiadiazol-3-yl; 1,2,4-thiadiazol-5-yl; isothiazol-3-yl; 1,2,3-triazol-4-yl; 1,2,3-triazol-5-yl; 1,2,4-triazin-3-yl; 1,3,5-triazin-2-yl; tetrazol-5-yl; isoxazol-3-yl; 1,2,3,4-oxatriazol-5-yl; 1,2,3-oxadiazol-4-yl; 1,3,4-oxadiazol-2-yl; 1,2,4-oxadiazol-3-yl; 2-pyrazolin-3-yl; pyrazol-3-yl; pyrazin-2-yl; pyridazin-3-yl; pyrimidin-2-yl; 1H-indazole-3-yl; benzoxazol-2-yl; benzimidazol-2-yl; benzothiazol-2-yl; 4,5,6,7-tetrahydro-benzothiazol-2-yl; cinnolin-3-yl; phthalazin-1-yl; naphtho[2,1-d]thiazol-2-yl; naphtho[1,2-d]thiazol-2-yl; quinoxalin-2-yl; 4-oxoquinazolin-2-yl; quinazolin-2-yl; quinazolin-4-yl; purin-2-yl; purin-8-yl; pteridin-2-yl; pteridin-6-yl; oxazolo[4, 5-1)]pyridin-2-yl; oxazolo[4,5-c]pyridin-2-yl; oxazolo[5,4-c]pyridin-2-yl; oxazolo[5,4-b]pyridin-2-yl; thiazolo[4,5-b]pyridin-2-yl; thiazolo[5,4-b]pyridin-2-yl and thiazolo[5,4-c]pyridin-2-yl. In particular examples, J is benzoxazolyl; 1,2,3-oxadiazol-4-yl; 1,3,4-oxadiazol-2-yl; 1,2,4-oxadiazol-3-yl; oxazolo[4, 5-1)]pyridin-2-yl, oxazolo[4,5-c]pyridin-2-yl, oxazolo[5,4-c]pyridin-2-yl or oxazolo[5,4-b]pyridin-2-yl, each of which is optionally substituted with C₁₋₆ alkyl, halo, cyclopropyl, SO₂(C₁₋₆alkyl), OCH₃, SO₂N(CH₃)₂, SO₂NH₂, CF₃ or —(CR₂)₁—R⁵.

In the above Formula (1), (2) and (3), R¹ is a non-basic substituent or the residue of a relatively weak base, having for example a pKa<5, a pKa<2, or a pKa<0. Examples of R¹ include but are not limited to H, an optionally halogenated C₁₋₆ alkyl, C₂₋₆ alkenyl or C₃₋₆ alkynyl; cyano, OH, O(CR₂)₁R⁶, SO₂R⁶, CONR(CR₂)₁R⁶, CONR⁷R⁸ or

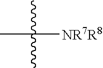

wherein R⁷ and R⁸ together with N in NR⁷R⁸ form an optionally substituted 5-7 membered heterocyclic ring; or R¹ is an optionally substituted C₃₋₇ cycloalkyl, aryl, or a 5-7 membered heterocyclic ring or heteroaryl having no nitrogen atoms; or

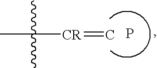

wherein ring P is an optionally substituted 5-7 membered carbocyclic ring.

In the above Formula (1), (2) and (3), each optionally substituted moiety may be substituted with halo, ═O, C₁₋₆ alkoxy, amino, C₁₋₆ alkyl, C₂₋₆ alkenyl or C₂₋₆ alkynyl, each of which may optionally be halogenated or may optionally have a carbon that may be replaced or substituted with N, O or S; CO₂R¹⁰, O—(CR₂)₁—C(O)—R¹⁰; —(CR₂)₁—R¹⁰, —(CR₂)₁—C(O)—R¹⁰, or —(CR₂)₁—SO₂—R¹⁰; or a combination thereof, wherein each R¹⁰ is H, amino, C₁₋₆ alkyl, or an optionally substituted carbocyclic ring, heterocyclic ring, aryl or heteroaryl.

The present invention also includes all suitable isotopic variations of the compounds of the invention, or pharmaceutically acceptable salts thereof. An isotopic variation of a compound of the invention or a pharmaceutically acceptable salt thereof is defined as one in which at least one atom is replaced by an atom having the same atomic number but an atomic mass different from the atomic mass usually found in nature. Examples of isotopes that may be incorporated into the compounds of the invention and pharmaceutically acceptable salts thereof include but are not limited to isotopes of hydrogen, carbon, nitrogen and oxygen such as ²H, ³H, ¹¹C, ¹³C, ¹⁴C, ¹⁵N, ¹⁷O, ¹⁸O, ³⁵S, ¹⁸F, ³⁶Cl and ¹²³I. Certain isotopic variations of the compounds of the invention and pharmaceutically acceptable salts thereof, for example, those in which a radioactive isotope such as ³H or ¹⁴C is incorporated, are useful in drug and/or substrate tissue distribution studies. In particular examples, ³H and ¹⁴C isotopes may be used for their ease of preparation and detectability. In other examples, substitution with isotopes such as ²H may afford certain therapeutic advantages resulting from greater metabolic stability, such as increased in vivo half-life or reduced dosage requirements. Isotopic variations of the compounds of the invention or pharmaceutically acceptable salts thereof can generally be prepared by conventional procedures using appropriate isotopic variations of suitable reagents.

The compounds and compositions of the invention may be useful for modulating a channel activating protease. Examples of channel activating proteases which may be modulated using the compounds and compositions of the invention include but are not limited to prostasin, PRSS22, TMPRSS11 (e.g., TMPRSS11B, TMPRSS11E), TMPRSS2, TMPRSS3, TMPRSS4 (MTSP-2), matriptase (MTSP-1), CAP2, CAP3, trypsin, cathepsin A, or neutrophil elastase. The compounds of this invention may also inhibit the activity of proteases that stimulate the activity of ion channels, such as the epithelial sodium channel, and may be useful in the treatment of CAP-associated diseases.

Pharmacology and Utility

Compounds of the invention modulate the activity of channel activating protease, particularly trypsin-like serine proteases such as prostasin, and as such, are useful for treating diseases or disorders in which prostasin, for example, contribute to the pathology and/or symptomology of the disease.

Diseases mediated by inhibition of a channel activating protease, particularly by a trypsin-like serine protease such as prostasin, include diseases associated with the regulation of fluid volumes across epithelial membranes. For example, the volume of airway surface liquid is a key regulator of mucociliary clearance and the maintenance of lung health. The inhibition of a channel activating protease will promote fluid accumulation on the mucosal side of the airway epithelium thereby promoting mucus clearance and preventing the accumulation of mucus and sputum in respiratory tissues (including lung airways). Such diseases include respiratory diseases such as cystic fibrosis, primary ciliary dyskinesia, chronic bronchitis, chronic obstructive pulmonary disease (COPD), asthma, respiratory tract infections (acute and chronic; viral and bacterial) and lung carcinoma. Diseases mediated by inhibition of channel activating proteases also include diseases other than respiratory diseases that are associated with abnormal fluid regulation across an epithelium, perhaps involving abnormal physiology of the protective surface liquids on their surface, for example xerostomia (dry mouth) or keratoconjunctivitis sire (dry eye). Furthermore, CAP regulation of ENaC in the kidney could be used to promote diuresis and thereby induce a hypotensive effect.

Chronic obstructive pulmonary disease includes chronic bronchitis or dyspnoea associated therewith, emphysema, as well as exacerbation of airways hyper reactivity consequent to other drug therapy, in particular other inhaled drug therapy. The invention is also applicable to the treatment of bronchitis of whatever type or genesis including, for example, acute, arachidic, catarrhal, croupus, chronic or phthinoid bronchitis.

Asthma includes intrinsic (non-allergic) asthma and extrinsic (allergic) asthma, mild asthma, moderate asthma, severe asthma, bronchitic asthma, exercise-induced asthma, occupational asthma and asthma induced following bacterial infection. Asthma also encompasses a condition referred to as “wheezy-infant syndrome,” which involves subjects less than 4 or 5 years of age who exhibit wheezing symptoms and diagnosed or diagnosable as “wheezy infants,” an established patient category of major medical concern and often identified as incipient or early-phase asthmatics.

The suitability of a channel activating protease inhibitor such as a prostasin inhibitor for the treatment of a disease mediated by inhibition of a channel activating protease, may be tested by determining the inhibitory effect of the channel activating protease inhibitor according to the assays described below and following methods known in the art.

In accordance with the foregoing, the present invention further provides a method for preventing or treating any of the diseases or disorders described above in a subject in need of such treatment, which method comprises administering to said subject a therapeutically effective amount of a compound of Formula (1), (2) or (3), or a pharmaceutically acceptable salt thereof. For any of the above uses, the required dosage will vary depending on the mode of administration, the particular condition to be treated and the effect desired. (See, “Administration and Pharmaceutical Compositions”, infra).

Administration and Pharmaceutical Compositions

In general, compounds of the invention will be administered in therapeutically effective amounts via any of the usual and acceptable modes known in the art, either singly or in combination with one or more therapeutic agents.

Channel activating protease inhibitors of the invention are also useful as co-therapeutic agents for use in combination with another therapeutic agent. For example, a channel activating protease inhibitor may be used in combination with an anti-inflammatory, bronchodilatory, antihistamine or anti-tussive, antibiotic or DNase therapeutic agent. The channel activating protease inhibitor and other therapeutic agent may be in the same or different pharmaceutical composition. The channel activating protease inhibitor may be mixed with the other therapeutic agent in a fixed pharmaceutical composition, or it may be administered separately, before, simultaneously with or after the other therapeutic agent. The combination may be useful particularly in the treatment of cystic fibrosis or obstructive or inflammatory airways diseases such as those mentioned hereinbefore, for example as potentiators of therapeutic activity of such drugs or as a means of reducing required dosaging or potential side effects of such drugs.

Suitable anti-inflammatory therapeutic agents include steroids, in particular glucocorticosteroids such as budesonide, beclamethasone dipropionate, fluticasone propionate, ciclesonide or mometasone furoate, or steroids described in international patent application WO 02/88167, WO 02/12266, WO 02/100879, WO 02/00679 (for example, Examples 3, 11, 14, 17, 19, 26, 34, 37, 39, 51, 60, 67, 72, 73, 90, 99 and 101), WO 03/35668, WO 03/48181, WO 03/62259, WO 03/64445, WO 03/72592, WO 04/39827 and WO 04/66920; non-steroidal glucocorticoid receptor agonists, such as those described in DE 10261874, WO 00/00531, WO 02/10143, WO 03/82280, WO 03/82787, WO 03/86294, WO 03/104195. WO 03/101932, WO 04/05229, WO 04/18429, WO 04/19935 and WO 04/26248; LTD4 antagonists such as montelukast and zafirlukast; PDE4 inhibitors such cilomilast (ARIFLO® GlaxoSmithKline), ROFLUMILAST® (Byk Gulden), V-11294A (Napp), BAY19-8004 (Bayer), SCH-351591 (Schering-Plough), AROFYLLINE® (Almirall Prodesfarma), PD189659/PD168787 (Parke-Davis), AWD-12-281 (Asta Medica), CDC-801 (Celgene), Se1CID(TM) CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204. WO 03/104205, WO 03/39544, WO 04/000814, WO 04/000839, WO 04/005258. WO 04/018450, WO 04/018451, WO 04/018457, WO 04/018465, WO 04/018431, WO 04/018449, WO 04/018450, WO 04/018451, WO 04/018457, WO 04/018465. WO 04/019944, WO 04/019945, WO 04/045607 and WO 04/037805; and adenosine A_{2B} receptor antagonists such as those described in WO 02/42298, each of which is incorporated herein in its entirety.

Suitable bronchodilatory therapeutic agents include beta-2 adrenoceptor agonists such as albuterol (salbutamol), metaproterenol, terbutaline, salmeterol fenoterol, procaterol, formoterol, carmoterol, or pharmaceutically acceptable salts thereof; and compounds (in free or salt or solvate form) of Formula (1) as described in WO 00/75114, a compound of formula:

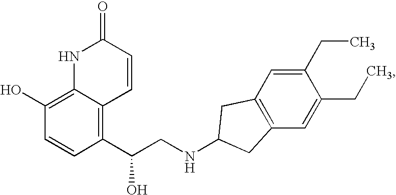

compounds of Formula (1) of WO 04/16601 (in free or salt or solvate form), and compounds of EP 1440966, JP 05025045, WO 93/18007, WO 99/64035, US 2002/0055651, WO 01/42193, WO 01/83462, WO 02/66422, WO 02/70490, WO 02/76933, WO 03/24439, WO 03/42160, WO 03/42164, WO 03/72539, WO 03/91204, WO 03/99764, WO 04/16578, WO 04/22547, WO 04/32921, WO 04/33412, WO 04/37768, WO 04/37773, WO 04/37807, WO 04/39762, WO 04/39766, WO 04/45618 WO 04/46083 and WO 04/80964 or pharmaceutically acceptable salts thereof, each of which is incorporated herein in its entirety.

Suitable bronchodilatory therapeutic agents also include anticholinergic or antimuscarinic agents, in particular ipratropium bromide, oxitropium bromide, tiotropium salts and CHF 4226 (Chiesi), and glycopyrrolate, but also those described in EP 424021, U.S. Pat. No. 3,714,357, U.S. Pat. No. 5,171,744, WO 01/04118, WO 02/00652, WO 02/51841, WO 02/53564, WO 03/00840, WO 03/33495, WO 03/53966, WO 03/87094, WO 04/018422 and WO 04/05285, each of which is incorporated herein in its entirety.

Suitable dual anti-inflammatory and bronchodilatory therapeutic agents include dual beta-2 adrenoceptor agonist/muscarinic antagonists such as those disclosed in US 2004/0167167, WO 04/74246 and WO 04/74812.

Suitable antihistamine therapeutic agents include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in JP 2004107299, WO 03/099807 and WO 04/026841, each of which is incorporated herein in its entirety.

Suitable antibiotics include macrolide antibiotics, for example tobramycin (TOBIT™).

Suitable DNase therapeutic agents include dornase alfa (PULMOZYME™), a highly purified solution of recombinant human deoxyribonuclease I (rhDNase), which selectively cleaves DNA. Dornase alfa is used to treat cystic fibrosis.

Other useful combinations of channel activating protease inhibitors with anti-inflammatory therapeutic agents are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, Takeda antagonists such as N-[[4-[[[6,7-dihydro-2-(4-methyl-phenyl)-5H-benzo-cyclohepten-8-yl]carbonyl]amino]phenyl]-methyl]tetrahydro-N,N-dimethyl-2H-pyran-4-amin-ium chloride (TAK-770), and CCR-5 antagonists described in U.S. Pat. No. 6,166,037, WO 00/66558, WO 00/66559, WO 04/018425 and WO 04/026873, each of which is incorporated herein in its entirety.

In the treatment of a disease mediated by inhibition of prostasin in accordance with the invention, a channel activating protease inhibitor of the invention, in free form or in pharmaceutically acceptable salt form, may be administered by any appropriate route, for example orally, e.g. in tablet, capsule or liquid form, parenterally, for example in the form of an injectable solution or suspension, or intranasally, for example in the form of an aerosol or other atomisable formulation using an appropriate intranasal delivery device, e.g. a nasal spray such as those known in the art, or by inhalation, particularly for use with a nebulizer.

The channel activating protease inhibitor may be administered in a pharmaceutical composition together with a pharmaceutically acceptable diluent or carrier. Such compositions may be, for example dry powders, tablets, capsules and liquids, but also injection solutions, infusion solutions or inhalation suspensions, which may be prepared using other formulating ingredients and techniques known in the art.

The dosage of the channel activating protease inhibitor in free form or in pharmaceutically acceptable salt form can depend on various factors, such as the activity and duration of action of the active ingredient, the severity of the condition to be treated, the mode of administration, the species, sex, ethnic origin, age and weight of the subject and/or its individual condition. A typical daily dose for administration, for example oral administration to a warm-blooded animal, particularly a human being weighing about 75 kg, is estimated to be from approximately 0.7 mg to approximately 1400 mg, more particularly from approximately 5 mg to approximately 200 mg. That dose may be administered, for example, in a single dose or in several part doses of, for example, from 5 to 200 mg.

When the composition comprises an aerosol formulation, it may contain, for example, a hydro-fluoro-alkane (HFA) propellant such as HFA134a or HFA227 or a mixture of these, and may contain one or more co-solvents known in the art such as ethanol (up to 20% by weight), and/or one or more surfactants such as oleic acid or sorbitan trioleate, and/or one or more bulking agents such as lactose. When the composition comprises a dry powder formulation, it may contain, for example, the channel activating protease inhibitor having a particle diameter up to 10 microns, optionally together with a diluent or carrier, such as lactose, of the desired particle size distribution and a compound that helps to protect against product performance deterioration due to moisture e.g. magnesium stearate. When the composition comprises a nebulised formulation, it may contain, for example, the channel activating protease inhibitor either dissolved, or suspended, in a vehicle containing water, a co-solvent such as ethanol or propylene glycol and a stabilizer, which may be a surfactant.

In particular embodiments, the invention provides compounds of Formula (1), (2) or (3) in inhalable form, e.g. in an aerosol or other atomisable composition or in inhalable particulate, e.g. micronised, form. The invention also provides an inhalable medicament comprising compounds of the invention in inhalable form; a pharmaceutical product comprising compounds of the invention in inhalable form in association with an inhalation device; and an inhalation device comprising compounds of the invention in inhalable form.

Processes for Making Compounds of the Invention

The compounds of the invention may be prepared, following procedures exemplified in the Examples.

In the reactions described, reactive functional groups, where desired in the final product (e.g., hydroxy, amino, imino, thio or carboxy groups), may be protected using protecting groups known in the art, to avoid their unwanted participation in the reactions. Conventional protecting groups may be used in accordance with standard practice, for example, see T. W. Greene and P. G. M. Wuts in “Protective Groups in Organic Chemistry”, John Wiley and Sons, 1991.

Compounds of the invention may also be prepared as a pharmaceutically acceptable acid addition salt by reacting the free base form of the compound with a pharmaceutically acceptable inorganic or organic acid. Alternatively, a pharmaceutically acceptable base addition salt of a compound of the invention may be prepared by reacting the free acid form of the compound with a pharmaceutically acceptable inorganic or organic base. Alternatively, salt forms of the compounds of the invention may be prepared using salts of the starting materials or intermediates.

The free acid or free base forms of the compounds of the invention may be prepared from the corresponding base addition salt or acid addition salt from, respectively. For example, a compound of the invention in an acid addition salt form may be converted to the corresponding free base by treating with a suitable base (e.g., ammonium hydroxide solution, sodium hydroxide, and the like). A compound of the invention in a base addition salt form may be converted to the corresponding free acid by treating with a suitable acid (e.g., hydrochloric acid, etc.).

Compounds of the invention in unoxidized form may be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention may be prepared by methods known to those of ordinary skill in the art (e.g., for further details see Saulnier et al., (1994), Bioorganic and Medicinal Chemistry Letters, Vol. 4, p. 1985). For example, appropriate prodrugs may be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention may be made by means known to those of ordinary skill in the art. A detailed description of techniques applicable to the creation of protecting groups and their removal may be found in T. W. Greene, “Protecting Groups in Organic Chemistry”, 3^{rd} edition, John Wiley and Sons, Inc., 1999.

Compounds of the present invention may be conveniently prepared, or formed during the process of the invention, as solvates (e.g., hydrates). Hydrates of compounds of the present invention may be conveniently prepared by recrystallization from an aqueous/organic solvent mixture, using organic solvents such as dioxin, tetrahydrofuran or methanol.

Compounds of the invention may be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers may be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and may be readily separated by taking advantage of these dissimilarities. The diastereomers may be separated by chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture may be found in Jean Jacques, Andre Collet, Samuel H. Wilen, “Enantiomers, Racemates and Resolutions”, John Wiley And Sons, Inc., 1981.

In summary, the compounds of the invention may be prepared as exemplified in the Examples, and Formula (1), (2) and (3) may be made by a process, which involves:

(a) optionally converting a compound of the invention into a pharmaceutically acceptable salt;

(b) optionally converting a salt form of a compound of the invention to a non-salt form;

(c) optionally converting an unoxidized form of a compound of the invention into a pharmaceutically acceptable N-oxide;

(d) optionally converting an N-oxide form of a compound of the invention to its unoxidized form;

(e) optionally resolving an individual isomer of a compound of the invention from a mixture of isomers;

(f) optionally converting a non-derivatized compound of the invention into a pharmaceutically acceptable prodrug derivative; and

(g) optionally converting a prodrug derivative of a compound of the invention to its non-derivatized form.

Insofar as the production of the starting materials is not particularly described, the compounds are known or may be prepared analogously to methods known in the art or as disclosed in the Examples hereinafter. One of skill in the art will appreciate that the above transformations are only representative of methods for preparation of the compounds of the present invention, and that other well-known methods may similarly be used. The present invention is further exemplified, but not limited, by the following intermediates (Reference compounds) and Examples that illustrate the preparation of the compounds of the invention.

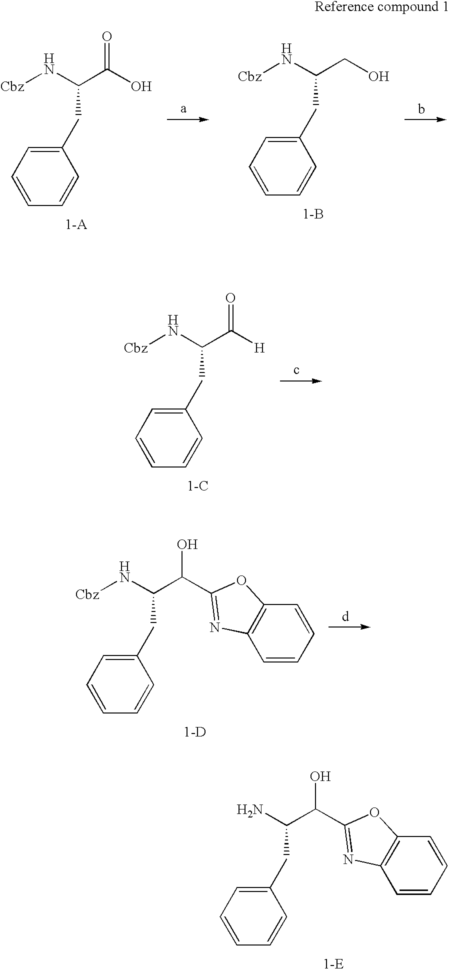

1-B: The starting material, Cbz-Phe-OH (15.0 g, 50.0 mmol) is dissolved in THF (150 mL) and the solution is cooled to −10° C. followed by the addition of triethylamine (7.1 ml, 50.0 mmol) and dropwise addition of isobutylchloroformate (7.1 ml, 55 mmol). The resulting suspension is stirred for two hours at 0° C. The reaction mixture is filtrated and cooled to −10° C. NaBH₄ (3.97 g, 105 mmol) is dissolved in water (50 ml) at 0° C., and the solution is added portionwise to the THF solution. The reaction mixture is allowed to warm to room temperature and stirred for one hour. The reaction mixture is acidified with 1N HCl solution and the aqueous phase is extracted several times with EtOAc. The combined organic layers are washed with water, saturated aqueous NaHCO₃ solution and brine; dried on MgSO₄; and the solvent is removed in vacuo. The product is purified by flash column chromatography (hexanes/ethyl acetate) to afford the desired product as a white foam.

1-C: The alcohol (12.02 g, 42.1 mmol) is dissolved in DCM (100 ml) and cooled to 0° C. A solution of the Dess-Martin reagent (19.5 g, 46.2 mmol) in DCM (100 ml) is added portionwise. The suspension is allowed to warm to room temperature and stirred until complete conversion (˜2 hr). A 1:1 mixture of saturated aqueous NaHCO₃ solution and a 1M Na₂S₂O₃ solution is added, and the resulting biphasic system is stirred vigorously for 20 minutes. The organic layer is separated and the aqueous layer is extracted one time with DCM. The combined organic layers are distilled in vacuo and the resulting oil is taken up in EtOAc; washed six times with the NaHCO₃/Na₂S₂O₃ mixture, water and brine; dried on MgSO₄; and the solvent is removed in vacuo to give the crude aldehyde as a yellowish oil. The material is directly used in the next step without further purification.

1-D: To a solution of iso-PrMgCl (70.3 mmol, 35 ml of a 2M-THF solution from Sigma-Aldrich) in THF (100 ml) is added benzoxazole (8.36 g, 703 mmol) in THF (20 ml) at −20° C. The reaction mixture is stirred at −20° C. for 30 minutes (color change: deep red) and a solution of the aldehyde (11.9 g, 42.0 mmol) in THF (20 ml) is added slowly under temperature control at −20° C. to −15° C. The reaction mixture is allowed to warm to room temperature and stirred until completion. The reaction is quenched with saturated aqueous NH₄Cl solution, and the solvent is removed in vacuo. The aqueous phase is extracted three times with EtOAc, and the combined organic layers are excessively washed with 1N HCl solution, water and brine; dried on MgSO₄; and the solvent is removed in vacuo to give the crude benzoxazole as a deep red oil. Purification on silica with EtOAc/hexanes (1:5 to 1:1) gives the benzoxazole as a pale yellow solid.

1-E: Compound 5 (1.6 g, 5.96 mmol) is dissolved in ethanol (3 mL). Pd/C (10%, wet, Degussa type) is added, and the flask is placed on a Parr shaker overnight and subjected to hydrogen gas at 40 psi. The catalyst is filtered through Celite, and the solvent is removed in vacuo. The crude material is purified by flash chromatography using first a gradient of hexanes/EtOAc to remove less polar and colored impurities, then followed by a gradient of DCM/MeOH to elute the desired compound 5. The solvent is removed in vacuo to afford the desired compound as a white solid.

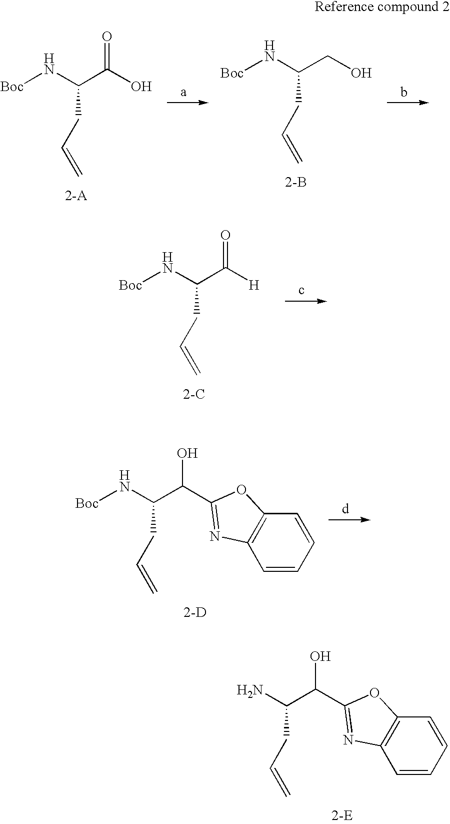

2-B: This compound is prepared from L-Boc-allylglycine using methods analogous to those described for the preparation Reference Compound 1-B.

2-C: This compound is prepared from Reference Compound 2-B using methods analogous to those described for the preparation Reference Compound 1-C.

2-D: This compound is prepared from Reference Compound 2-C using methods analogous to those described for the preparation Reference Compound 1-D.

2-E: Reference Compound 2-D (350 mg, 1.10 mmol) is dissolved in methylene chloride (3 mL). TFA (2 mL) is added, and the reaction is stirred at room temperature until the starting material is consumed. The solvent is removed in vacuo to afford the product 2-E as the TFA salt which is used without further purification.

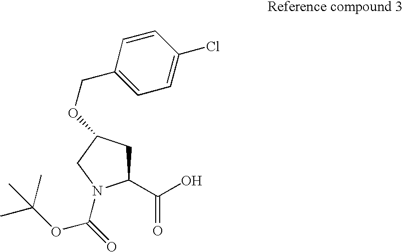

Finely powdered KOH (19.4 g, 0.346 mol) is dissolved in DMSO and stirred at room temperature for 20 min and then cooled to 0° C. N-Boc-trans-4-hydroxy-L-proline (Boc-Hyp-OH) (10 g, 43.3 mmol) is dissolved in DMSO (10 mL) and added, and the reaction mixture is stirred for an additional 10 min at 0° C. Next, 4-chlorobenzyl chloride (33.0 g, 0.204 mol) is added, and the reaction mixture is stirred at 0° C. for an additional 15 min, after which the ice bath is removed and the reaction mixture is allowed to warm to room temperature and stir for 4 h. The reaction mixture is poured into water (300 mL), and the reaction vessel is rinsed with an additional aliquot of water (300 mL). The combined aqueous layer is extracted with ether (2×300 mL) and discarded. The aqueous layer is acidified with 87% H₃PO₄ to pH 2.3 and then extracted with ether (3×300 mL). The combined ether extracts are washed with water (2×400 mL) and brine (2×400 mL) and then dried over MgSO₄, filtered and concentrated in vacuo. The residue is purified by chromatography on silica gel with EtOAc/Hexanes (gradient 0 to 100%) to give compound 3 as a clear oil. MS m/z 256.1 (M+1−Boc); ¹H NMR (DMSO-D₆, 400 MHz) δ 7.39-7.31 (4H, m), 4.52-4.40 (2H, m), 4.16-4.10 (2H, m), 3.48-3.41 (2H, m), 2.40-2.30 (1H, m), 2.03-1.94 (1H, m), 1.39-1.34 (9H, m).

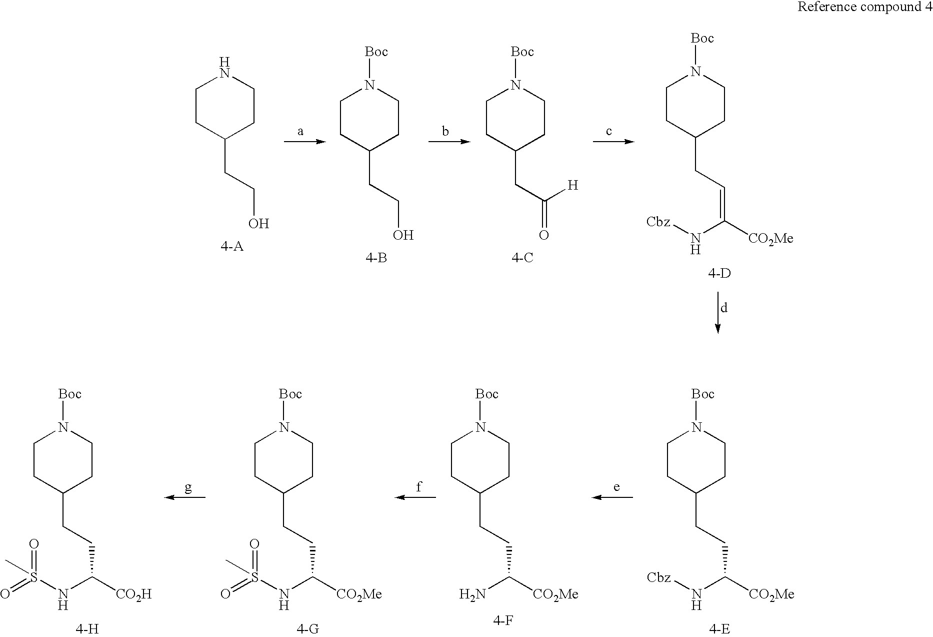

4-B: 4-piperidine ethanol (4-A) (5 g, 39.7 mmol) is dissolved in THF (120 mL). Triethylamine (5.6 mL, 40 mmol) is added and the solution is cooled to 0° C. Boc₂O (9.59 g, 44 mmol) is added and the reaction is stirred overnight at room temperature. Solvent is removed in vacuo, the crude residue dissolved in ethyl acetate (120 mL) is added, and the solution is washed with 0.1 N HCl (3×100 mL) and brine (1×100 mL), dried with MgSO₄, filtered and solvent evaporated in vacuo to give compound 4-B as a clear oil.

4-C: Trichloroisocyanuric acid (2.66 g, 11.46 mmol) is added to a solution of the alcohol (2.39 g, 10.42 mmol) in DCM, and the solution is stirred and maintained at 0° C., followed by addition of a catalytic amount of TEMPO. After the addition, the mixture is warmed to room temperature and stirred for an hour and then filtered on Celite. The organic phase is washed with saturated aqueous Na₂CO₃, followed by 1N HCL and brine. The organic layer is dried (MgSO₄) and the solvent is evaporated to give 4-C. ¹H NMR (CDCl₃, 400 MHz) δ 9.72 (1H, s), 4.07-4.01 (2H, m), 2.70-2.57 (2H, m), 2.35-2.31 (2H, m), 2.05-1.94 (1H, m), 1.64-1.46 (2H, m), 1.39 (9H, s), 1.30-1.02 (2H, m).

4-D: To a solution of Cbz-α-phosphonoglycine trimethyl ester, (2.8 g, 8.45 mmol) in THF at −78° C. is added 1,1,3,3-tetramethyl-guanidine (1.022 ml, 8.14 mmol). After 10 minutes, the aldehyde 3 (1.76 g, 7.76 mmol) is added. The solution is then placed in an ice bath at 0° C. for 1 hour, warmed to room temperature and stirred for one more hour. The solution is diluted with EtOAc, washed with 1M NaHSO₄, dried (MgSO₄) and concentrated in vacuo. The residue is purified by chromatography (ISCO) with Ethyl acetate/Hexane 0 to 100% to afford 4-D as a white solid. MS m/z 333.2 (M+1), ¹H NMR (CDCl₃, 400 MHz) δ. 7.35-7.33 (5H, m), 6.63 (1H, t, J=8 Hz), 6.30 (1H, bs), 5.12 (2H, s), 4.10-4.04 (2H, m), 3.73 (3H, s), 2.67-2.62 (2H, m), 2.14 (2H, t, J=6.8 Hz), 1.63-1.46 (3H, m), 1.43 (9H, s), 1.14-1.06 (2H, m).

4-F (steps d and e): A Parr vessel is charged with 4-D (1 g, 2.31 mmol) and MeOH (100 ml) under nitrogen. The solution is subjected to three cycles of vacuum and nitrogen bubbling, and the catalyst (R,R)-Ethyl-DuPHOS-Rh(COD) triflate is added (30 mg, 0.04 mmol). The mixture is placed under 60 psi of hydrogen gas at room temperature for 24 h. The conversion to 4-E is complete after 24 h, and is used in the next step (e) without isolation. The solution is flushed with nitrogen and Pd/C (5% wt) is added. The mixture is placed under 50 psi of H₂ at rt for another 24 h. The mixture is flushed with nitrogen and filtered on Celite. The Celite cake is washed with MeOH and the organic solution is concentrated under vacuum. Hexanes are added and then evaporated in vacuo to azeotrope the remaining methanol to afford 4-F as an oil, which is then used in the next step without further purification.

4-G: Crude 4-F (0.6 g, 1.99 mmol) is dissolved in THF (10 mL), and 2,4,6-collidine (315 mg, 2.38 mmol) and methanesulfonyl chloride (0.170 ml, 2.19 mmol) are added to the solution and stirred for 2 hours. The reaction is diluted with EtOAc (50 mL); washed with 1M NaHSO₄ (2×25 mL) and brine (25 mL); and dried (MgSO₄). The solvent is removed in vacuo and the crude residue purified by flash chromatography using a gradient of hexanes and EtOAc to give the desired product 4-G.

4-H: Compound 4-G (0.70 g, 1.84 mmol) is dissolved in dioxane (7 mL) and LiOH.H₂O (232 mg, 5.55 mmol) dissolved in water (4 mL) is added. The reaction mixture is stirred for 1 h. The solvent is evaporated, and the residue diluted with EtOAc (25 mL), washed with 1N NaHSO₄ (25 mL) and brine (25 mL), and dried (MgSO₄). The solvent is removed in vacuo, and the crude purified by silica gel chromatography (Hexanes/EtOAc gradient) to afford Reference Compound 4 as a white solid.

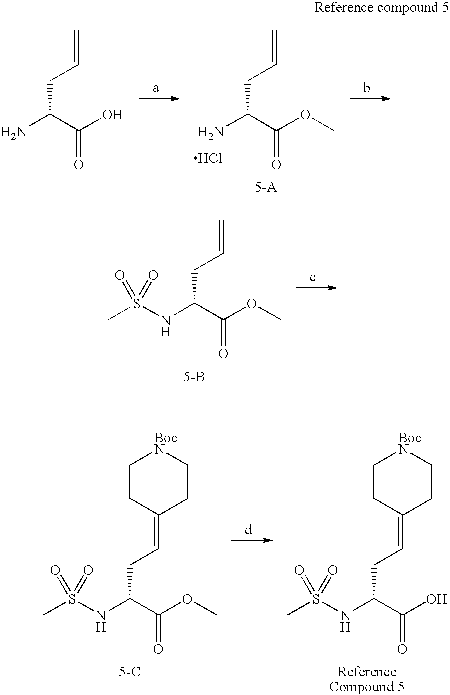

In the reaction scheme for Reference compound 3, the reagents and conditions are: (a) SOCl₂ (3.0 equiv.), MeOH, 0° C., 100%; (b) Mesyl chloride (1.2 equiv.), Et₃N (3.0 equiv.), cat. DMAP, THF, 23° C., 79%; (c) Hoveyda-Grubbs metathesis catalyst (8 mol %), N-Boc-4-methylenepiperidine (3.0 equiv.), DCM, 40° C., 51%; (d) LiOH, dioxanes, H₂O, 23° C., 100%.

5-A: D-allylglycine (5.03 g, 43.73 mmol, 1.0 equiv) is slurried in a suspension of methanol (70 mL) in an ice-water bath. Thionyl chloride (9.6 mL, 131.19 mmol, 3.0 equiv.) is added dropwise over 10 minutes. The reaction is warmed to room temperature until complete as shown by LC/MS. The solvent is evaporated and the resulting white solid of 5-A is directly used in the next step.

5-B: D-allylglycine methyl ester hydrochloride (5-A, 7.20 g, 43.73 mmol), Et₃N (18 mL, 131.19 mmol, 3.0 equiv.) and DMAP (10 mg, catalytic) are dissolved in THF (110 mL) and stirred at room temperature. Mesyl chloride (4.0 mL, 52.48 mmol, 1.2 eq.) is added dropwise, and the reaction stirred for 6 h at room temp. THF is evaporated and the crude reaction product dissolved in EtOAc (100 mL), washed with water (100 mL), 1N HCl (2×100 mL) and brine (100 mL), and dried (MgSO₄). The solvent is removed in vacuo and the crude material purified with flash chromatography (Hexanes:EtOAc) to give 5-B as a yellow oil.

5-C: Anhydrous dichloromethane (10 mL, 0.1 M) is added via syringe to 5-B (2.15 g, 10.37 mmol, 1.0 equiv.), and Hoveyda-Grubbs 2^{nd} Generation metathesis catalyst (1,3-Bis-(2,4,6-trimethylphenyl)-2-imidazolidinylidene) dichloro(o-isopropoxyphenylmethylene) ruthenium II dichloride) (510 mg, 0.815 mmol, 8 mol %) under a nitrogen atmosphere. N-Boc-4-methylenepiperidine (6 mL, 31.11 mmol, 3.0 eq.) is added via syringe and the reaction is fitted with a reflux condenser and heated to 40° C. for 12 hours. After the reaction is complete as shown by LC/MS, the reaction mixture is directly purified by automated silica-gel purification (0-100% ethyl acetate in hexanes) to provide 5-C as a dark green oil. MS m/z 277.2 (M−Boc+1).

Reference Compound 5: The saponification of 5-C is accomplished using the procedure previously described for the preparation of Reference Compound 4 (step g).

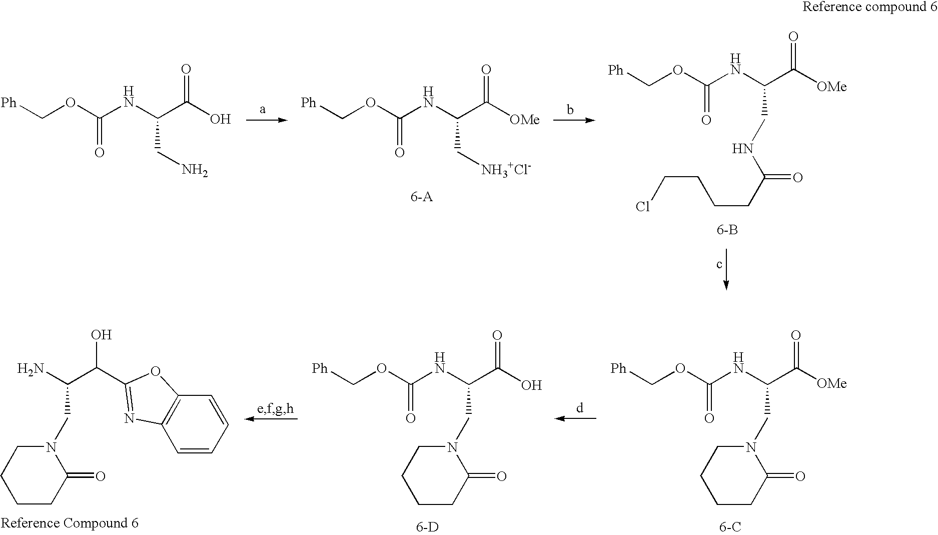

6-A: Thionyl chloride (9.1 mL, 125 mmol) is slowly added to methanol (250 mL) with stirring at 0° C. in an ice bath. After stirring for 30 minutes, N-alpha-Cbz-L-2,3-diaminopropionic acid (Z-Dap-OH) (15 g, 63 mmol) is added and the reaction is stirred overnight at room temperature. The solvent is removed in vacuo, and the resulting white solid is triturated in ether (300 mL) and filtered to give the methyl ester 6-A as the hydrochloride salt.

6-B: Reference compound 6-A (5.0 g, 17.4 mmol) is taken up in CH₂Cl₂ (70 mL) and cooled to 0° C. To this solution is added triethylamine (5.4 ml, 39.0 mmol), followed by 5-chlorovaleroyl chloride (2.63 g, 18.7 mmol). The reaction mixture is allowed to warm to room temperature and stirred for 4 h. The reaction is poured over brine (100 mL), and the organic layer extracted with DCM (2×50 mL), washed with 1N HCl, and dried (MgSO₄). Solvent is removed in vacuo and the crude material is purified by flash chromatography (hexanes/EtOAc) to give the desired product as a clear oil.

6-C: Chloride 6-B (4.1 g, 11.1 mmol) is dissolved in DMF (110 mL) and cooled to 0° C. To this solution is added NaH (0.53 g of 60% dispersion in mineral oil, 13.3 mmol), and the mixture is stirred at room temperature for 4 h. The DMF is removed in vacuo and the residue taken up in EtOAc; washed with 1 N HCl, saturated NaHCO₃ and brine; and dried over MgSO₄. The crude material is purified by silica gel chromatography to give lactam 6-C as a clear oil.

6-D: The saponification of 6-C is accomplished using the procedure previously described for the preparation of Reference Compound 4 (step g).

Reference compound 6 (steps e, f, g, and h): The conversion of 6-D to Reference Compound 6 is accomplished using the procedures previously described for the preparation of Reference Compound 1.

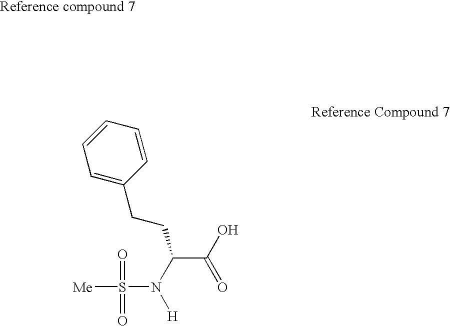

D-Homophenylalanine ethyl ester hydrochloride (5.00 g, 20.5 mmol) and DIEA (8.7 mL, 51.25 mmol) are dissolved in THF (100 mL) and stirred at room temperature. Mesyl chloride (1.67 mL, 21.52 mmol) is added dropwise, and the reaction stirred for 6 h at room temp. The THF is evaporated, and the crude dissolved in EtOAc (100 mL) and washed with water (100 mL), 1N HCl (2×100 mL) and brine (100 mL), and dried (MgSO₄). The solvent is removed in vacuo and the crude material purified with flash chromatography (Hexanes:EtOAc) to give the ethyl ester. The resulting ethyl ester is dissolved in dioxane (50 mL) and stirred at room temperature. LiOH.H₂O (1.00 mg, 24 mmol) dissolved in water (20 mL) is added, and the reaction stirred until the ethyl ester had disappeared (by TLC and LCMS). The solvent is removed in vacuo and the crude material is partitioned with EtOAc (50 mL) and 1N HCl (50 mL). The aqueous layer is extracted with EtOAc (2×50 mL) and the combined organic phases are washed with 1M NaHSO₄ (2×50 mL) and brine (50 mL), and dried with MgSO₄. The solvent is evaporated and the crude material purified by flash chromatography (EtOAc:Hexanes gradient) to give Reference Compound 7 as a white powder.

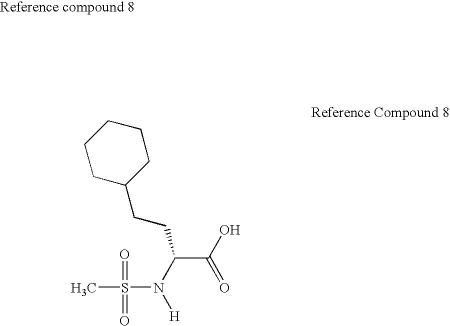

This compound is prepared starting from D-homocyclohexylalanine ethyl ester hydrochloride using methods analogous to those described for the preparation of Reference Compound 7.

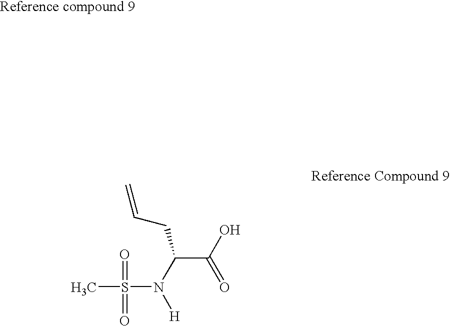

This compound is prepared starting from 3-cyanophenylalanine using methods analogous to those described for the preparation of Reference Compound 7.

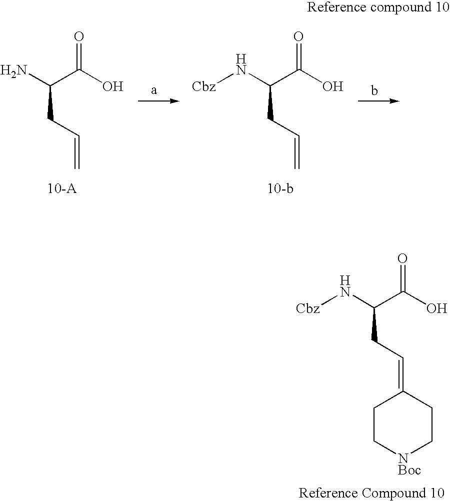

In the reaction scheme for Reference compound 10, the reagents and conditions are: (a) Cbz-OSu, Et₃N, THF, Water, 76%; (b) Hoveyda-Grubbs metathesis catalyst, N-Boc-4-methylenepiperidine, DCM, 40° C., 47%.

10-B: D-allylglycine (2.07 g, 18.0 mmol) and N-(benzyloxycarbonyloxy)succinimide (Cbz-OSu) (4.49 g, 18.0 mmol) are added to a round bottomed flask containing THF (60 mL) and water (20 mL). The mixture is stirred at room temperature and Et₃N (10.1 mL, 72.0 mmol) is added, and the reaction is stirred overnight at room temperature. The clear solution is diluted with EtOAc (200 mL), washed with 1N HCl (3×100 mL) and brine (1×100 mL), and dried with MgSO₄. Solvent is evaporated in vacuo to afford 10-B as a white solid, which is used without further purification.

Reference Compound 10: Anhydrous dichloromethane (4 mL, 0.2 M) is added via syringe to 10-B (193 mg, 0.766 mmol, 1.0 eq.), and Hoveyda-Grubbs 2nd Generation metathesis catalyst (1,3-Bis-(2,4,6-trimethylphenyl)-2-imidazolidinylidene)dichloro(o-isopropoxyphenylmethylene)ruthenium II dichloride) (98 mg, 0.115 mmol, 15 mol %) under a nitrogen atmosphere. N-Boc-4-methylenepiperidine (604 mg, 3.06 mmol, 4.0 eq.) is added via syringe and the reaction is fitted with a reflux condenser and heated to 40° C. for 12 hours. After the reaction is complete as shown by LC/MS, the reaction mixture is directly purified by automated silica-gel purification (0-100% ethyl acetate in hexanes) to provide Reference Compound 10 as a dark green oil. MS m/z 422.3 (M−Boc+1).

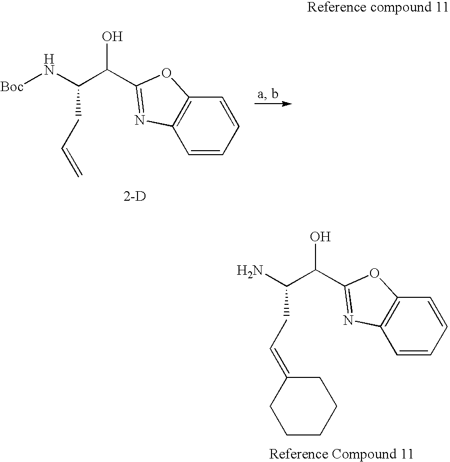

The cross-metathesis of 2-D with methylenecyclohexane is accomplished using methods analogous to those employed for the synthesis of Reference compound 10. The subsequent Boc deprotection is accomplished using methods analogous to those employed in the synthesis of 2-E, to provide Reference Compound 11.

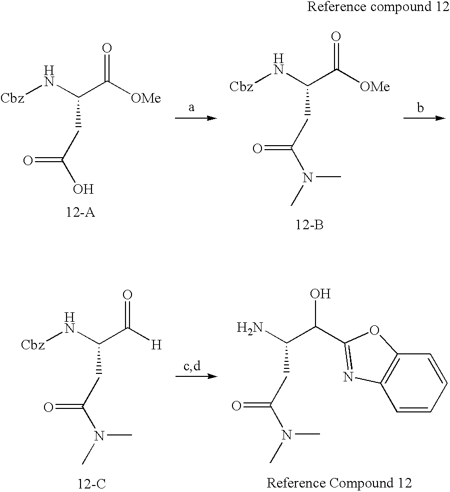

12-B: Cbz-Asp-OMe (2.5 g, 8.89 mmol) is dissolved in DCM (50 mL), dimethylamine hydrochloride (797 mg, 9.78 mmol) and HATU (3.72 g, 9.78 mmol) are added, and the solution is stirred at room temp for 10 min. Next is added DIEA (3.8 mL, 22.23 mmol), and the reaction mixture is allowed to stir overnight at room temperature. The solvent is removed in vacuo, and the crude material is directly purified by flash chromatography (hexanes/EtOAc gradient). The solvent is removed in vacuo to afford reference compound 12-B.

12-C: To a cold (−78° C.) solution of methyl ester 12-B (2.71 g, 8.8 mmol) in 100 mL of anhydrous DCM is added dropwise a 1 M solution of DiBAL-H in hexane (22 mL, 21.1 mmol) while keeping the reaction temperature below −70° C. The resulting solution is stirred at −78° C. for 1 h, and 5% aqueous citric acid (60 mL) is added to the reaction mixture. The mixture is stirred at room temperature for 10 min and the layers are then separated. The aqueous layer is extracted twice with DCM. The combined DCM solution is washed with water, dried over Na₂SO₄, and filtered. The filtrate is concentrated to afford the aldehyde 12-C, which is used directly in the next step without further purification.

The conversion of 12-C to Reference Compound 12 is accomplished using the procedures previously described for the preparation of Reference Compound 1 (steps c and d).

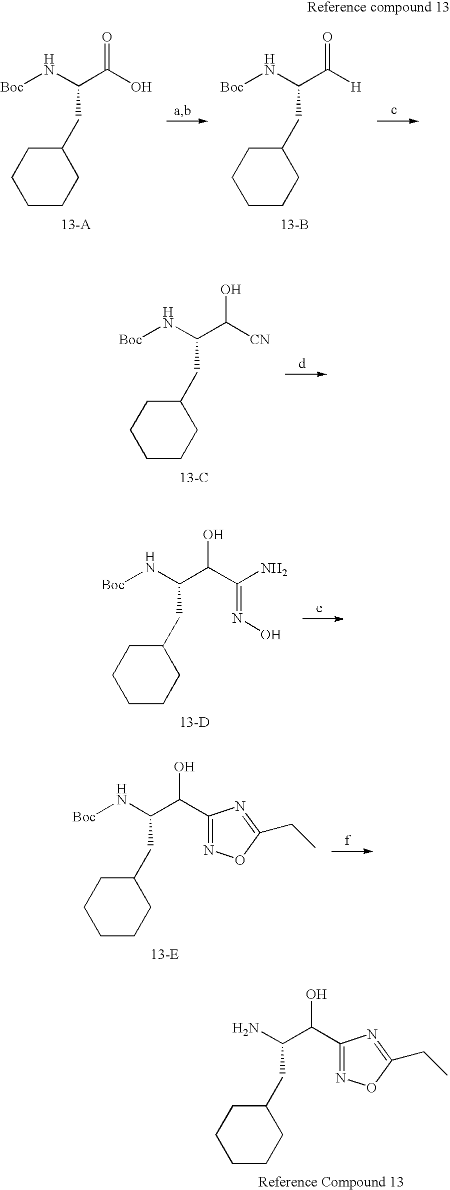

13-B (steps a and b): The conversion of commercially available Boc-Cha-OH to aldehyde 13-B is accomplished using the procedures previously described for the preparation of Reference Compound 1-C (steps a and b).

13-C: Aldehyde 13-B (5.06 g, 19.8 mmol) dissolved in dioxane (10 mL) is added to a cold (−5° C.) solution of sodium bisulfite (2.07 g, 19.8 mmol) in water (10 mL). KCN (1.29 g, 19.8 mmol) dissolved in water (5 mL) is added, and the reaction is allowed to gradually warm to room temperature while stirring overnight. The reaction is concentrated in vacuo and then diluted with water. The pH is adjusted to 5 with 1M NaHSO₄, and the aqueous phase is extracted with EtOAc. The combined organic layer is dried with Na₂SO₄ and concentrated. The crude material is purified by flash chromatography (hexanes/EtOAc) to afford the cyanohydrin 13-C.

13-D: Cyanohydrin 13-C (3.61 g, 12.8 mmol) is dissolved in EtOAc (50 mL) and treated with 50% aqueous hydroxylamine (1 mL). The solution is stirred and heated to 60° C. for 2 h, at which point the reaction is complete by LCMS. The solvent is removed in vacuo and the resulting crude material is used directly in the next step without further purification.

13-E: Hydroxylamidine 13-D (1.0 g, 3.17 mmol) is dissolved in dioxane (10 mL) in a SMITH PROCESS VIAL™. Propionic anhydride (0.45 mL, 3.49 mmol) is added and the solution is placed in a microwave reactor (e.g., Personal Chemistry Emrys Optimizer microwave reactor) and heated to 150° C. for 35 min. The solvent is removed in vacuo, and the crude residue purified by silica gel chromatography (hexanes/EtOAc as eluent) to give the oxadiazole 13-E.

Reference compound 13: (515 mg, 1.46 mmol) is dissolved in methylene chloride (30 mL). TFA (20 mL) is added and the reaction is stirred at room temperature until the starting material is consumed. The solvent is removed in vacuo, azeotroped with hexanes, and evaporated to dryness to afford the product as the TFA salt which is used without further purification.

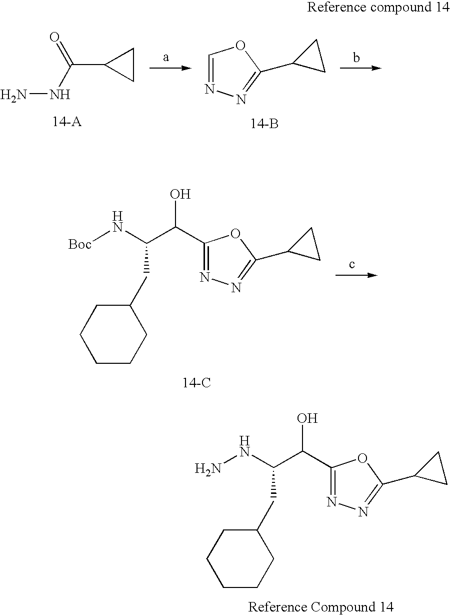

14-B: Commercially available cyclopropanecarboxylic acid hydrazide 14-A (1.0, 10.0 mmol) is added to trimethyl orthoformate (10 mL). p-Toluenesulfonic acid monohydrate (5 mg) is added and the reaction mixture is stirred and heated to reflux overnight. Solvent is removed in vacuo and the resulting crude residue is vacuum distilled to give 2-cyclopropyl-[1,3,4]oxadiazole 14-B.

14-C: A solution of 2-cyclopropyl-[1,3,4]oxadiazole 14-B (257 mg, 2.33 mmol) in anhydrous THF (12 mL) is cooled to −78° C. n-BuLi (1.6M in hexanes, 1.46 mL, 2.33 mmol) is added dropwise and the reaction mixture is stirred at −78° C. for 40 min MgBr₂.OEt (603 mg, 2.33 mmol) is added, and the reaction is allowed to warm to −45° C. and then stirred at that temperature for 90 min A solution of aldehyde 13-B (595 mg, 2.33 mmol) in THF (5 mL) is added, and the reaction is allowed to warm to −20° C. and stirred for an additional 4 h. The reaction is quenched with saturated aqueous NH₄Cl and then extracted with EtOAc. The combined organic extracts are washed with brine and dried over MgSO₄. The solvent is removed in vacuo, and the crude residue is purified by silica gel chromatography (hexanes/EtOAc) to afford the desired product 14-C.

Reference Compound 14: The deprotection of 14-C is accomplished using the procedures previously described for the preparation of Reference Compound 13 (step f).

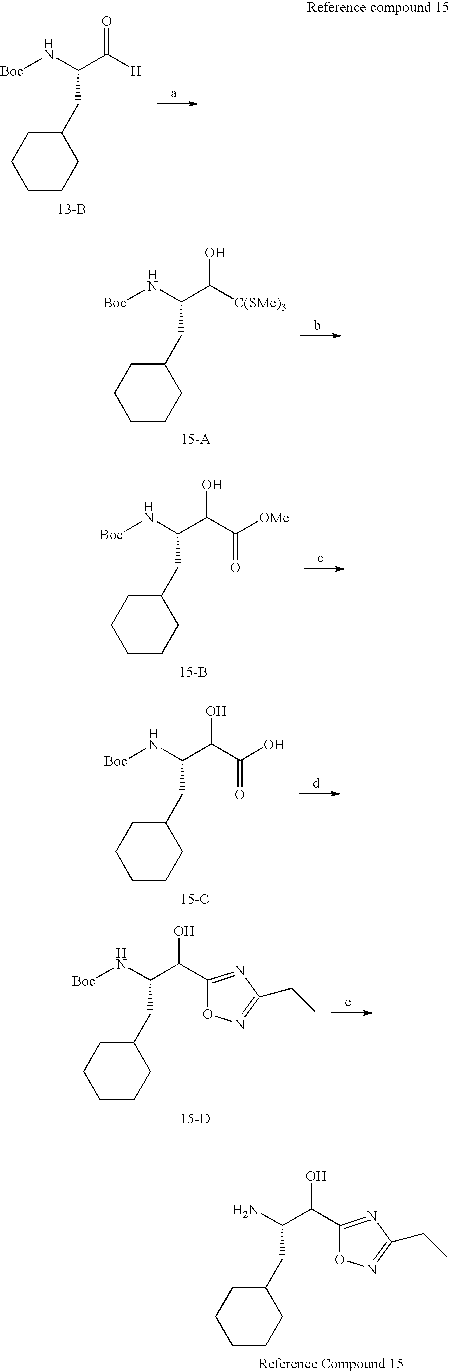

15-A: n-Butyllithium (2.5 M in hexanes, 19.8 mL, 49.7 mmol) is added over a period of 10 min to a stirred solution of tris(methylthio)methane (7.0 mL, 49.7 mmol) in THF (135 mL) at −65° C. After 20 min a precipitate forms, and a precooled (−65° C.) solution of aldehyde 13-B (2.96 g, 11.6 mmol) in THF (50 mL) is added over 30 min, upon which the precipitate dissolves. Stirring is continued for 5 h at −65° C. The reaction mixture is then poured into a stirred mixture of saturated aqueous NH₄Cl/DCM (400 mL, 1:12). The layers are separated, and the aqueous layer is extracted with DCM (3×100 mL). The combined organic phases are washed with water and brine, dried (MgSO₄), filtered, and evaporated to dryness in vacuo. Purification of the crude product by silica gel chromatography affords 15-A as an oil.

15-B: A solution of orthothioester 15-A (0.988 g, 2.41 mmol) in MeOH (46 mL) and water (4 mL) is stirred with HgCl₂ (2.20 g, 8.10 mmol) and HgO (0.658 g, 3.04 mmol) for three days. The reaction mixture is filtered over Celite, and the residue is washed with DCM (300 mL), MeOH (50 mL), and water (50 mL). The biphasic filtrate is separated, and the aqueous layer is extracted with DCM (3×50 mL). The combined organic phases are washed with saturated aqueous NH₄OAc (3×100 mL) and saturated aqueous NH₄Cl (2×100 mL), dried (Na₂SO₄), filtered and concentrated in vacuo. The resulting crude oil is purified by silica gel chromatography to afford methyl ester 15-B.

15-C: To a stirred solution of methyl ester 15-B (302 mg, 0.96 mmol) in THF/MeOH/H₂O (20 mL/5 mL/5 mL) is added powdered LiOH.H₂O (112 mg, 2.67 mmol). After 15 min, aqueous 1 M NaHSO₄ (8 mL) is added, and the solvent is evaporated under reduced pressure. The residue is diluted with H₂O (10 mL), acidified to pH=2 with aqueous 1M NaHSO₄, and extracted with EtOAc (3×10 mL). The combined organic phases are washed with H₂O and brine, dried (Na₂SO₄), filtered and concentrated in vacuo to afford the acid 15-C.

15-D: Acid 15-C (50 mg, 0.16 mmol) is dissolved in DCM (5 mL). N-hydroxypropionamidine (15 mg, 0.16 mmol) and DCC (34 mg, 0.16) are added, and the reaction is stirred for 2 h. The dicyclohexylurea byproduct is filtered, and the solvent removed under reduced pressure. The residue is dissolved in THF (5 mL), transferred to a SMITH PROCESS VIAL™, placed in a Personal Chemistry Emrys Optimizer microwave reactor and heated to 180° C. for 10 min The solvent is removed in vacuo and the crude residue purified by silica gel chromatography (hexanes/EtOAc as eluent) to afford oxadiazole 15-D.

Reference Compound 15: The deprotection of 15-D is accomplished using the procedures previously described for the preparation of Reference Compound 13 (step f).

### EXAMPLE 1

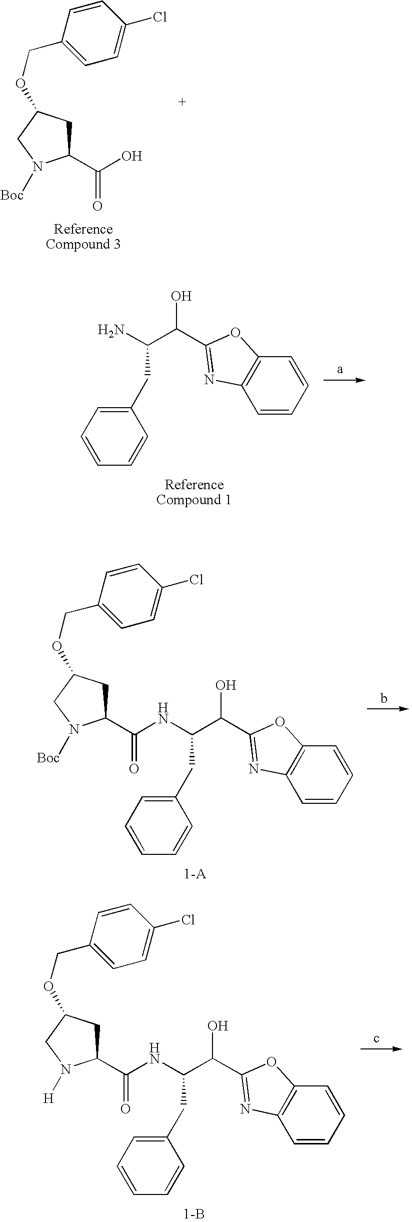

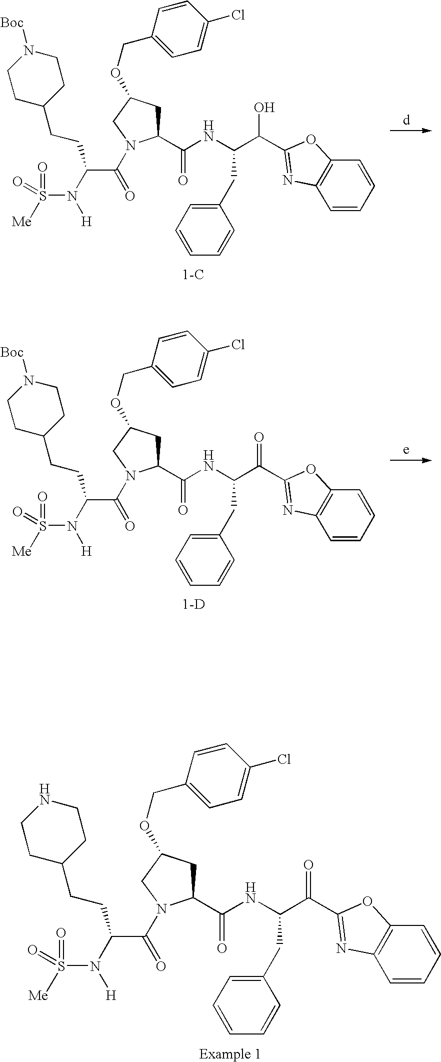

1-A: Reference Compound 1, as the TFA salt, (895 mg, 3.34 mmol) is dissolved in CH₂Cl₂ (50 mL). Reference Compound 3 (1.30 g, 3.67 mmol) and HATU (1.40 g, 3.67 mmol) are added, and the solution is stirred at room temp for 10 min. DIEA (1.5 mL, 8.4 mmol) is added via syringe and the reaction mixture is allowed to stir overnight at room temperature. The solvent is removed in vacuo, and the crude material is directly purified by flash chromatography (100 g silica, hexanes/EtOAc gradient). The solvent is removed in vacuo to afford 1-A as a foam.

1-B: A 20 mL screwcap vial is charged with a stirbar and 1-A (100 mg, 0.16 mmol). TFA (20%) in DCM (5 mL) is added, the vial is closed, and the solution is stirred for 1 h at room temperature. The solvent is removed in vacuo, hexanes is added and then evaporated again in vacuo to dryness, and repeated if necessary to azeotrope remaining TFA. The crude material is used directly in the next step without further purification.

1-C: Compound 1-B as the TFA salt (99 mg, 0.16 mmol) is dissolved in CH₂Cl₂ (5 mL). Reference Compound 4 (55 mg, 0.152 mmol) and HATU (67 mg, 0.176 mmol) are added, and the solution is stirred at room temp for 10 min DIEA (0.1 mL, 0.19 mmol) is added via syringe, and the reaction mixture is allowed to stir overnight at room temperature. The solvent is removed in vacuo, and the crude material is directly purified by flash chromatography (hexanes/EtOAc gradient). The solvent is removed in vacuo to afford 1-C as a foam.

1-D: Alcohol 1-C (113 mg, 0.13 mmol) is dissolved in DCM (10 mL) and Dess-Martin periodinane (66 mg, 0.15 mmol) is added. The reaction mixture is stirred overnight at room temperature. The solvent is removed in vacuo and the crude is purified by flash chromatography using a gradient of EtOAc:Hexanes to afford the ketone as a white foam.

Example 1: Ketone 1-D (59 mg, 0.069 mmol) is dissolved in DCM (1 mL) and TFA 50% in DCM (5 mL) is added. The reaction is stirred at room temp for 2 h and the solvent is removed in vacuo. The crude material is purified by reverse-phase HPLC, and the solvent is lyophilized to afford example 1 as a white powder.

### EXAMPLES 2-74

Examples 2-74 are prepared following methods analogous to Example 1, using appropriate acid and amine components that would be readily apparent to those skilled in the art.

### EXAMPLE 75

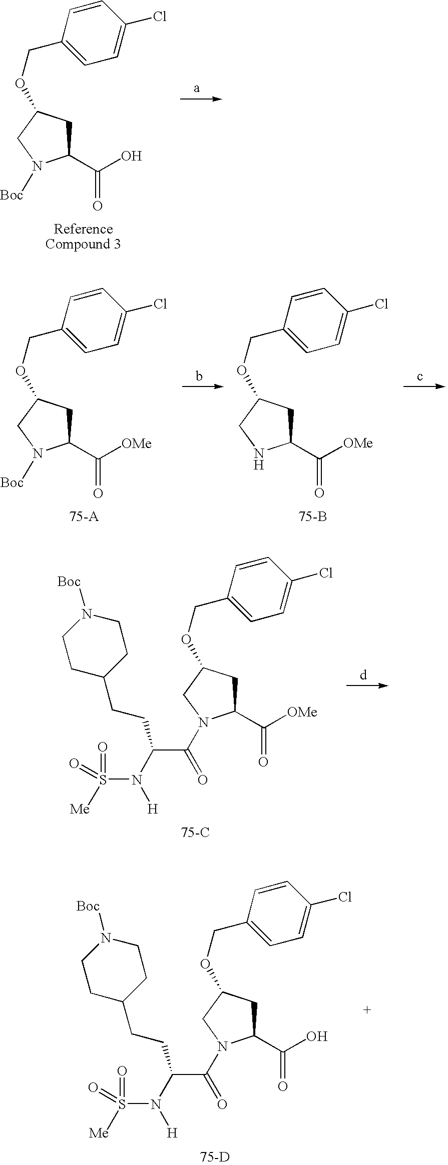

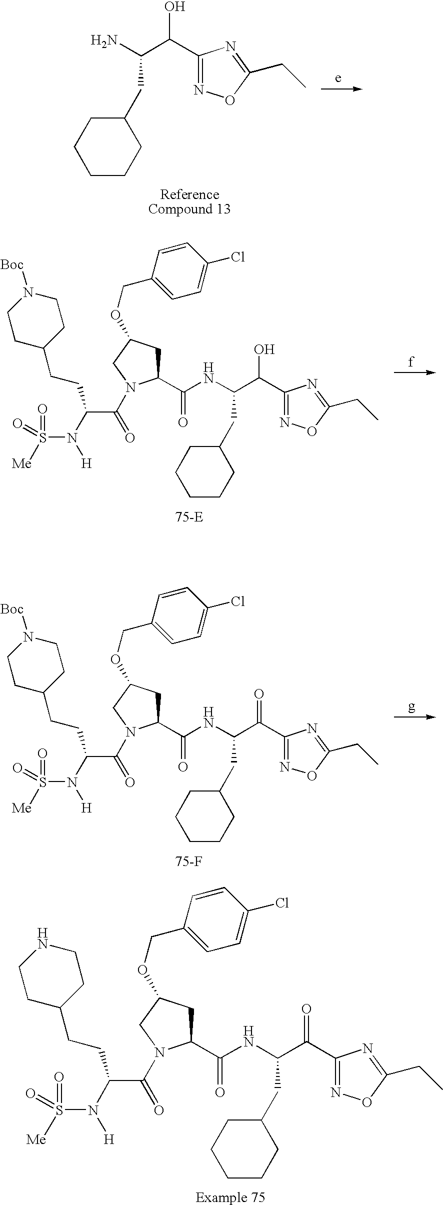

75-A: A solution of (trimethylsilyl)diazomethane (2M in diethylether) (4.7 ml, 9.45 mmol) is added to Reference compound 3 (2.4 g, 8.6 mmol) dissolved in CH₂Cl₂/MeOH 5:1 (25 mL) at room temperature. When the starting material had been consumed, as determined by LC/MS, the reaction mixture is quenched with acetic acid, concentrated in vacuo, and the crude residue is purified by flash chromatography (gradient EtOAc:Hexanes) to afford the methyl ester 75-A as a clear oil.

75-B: A round bottomed flask is charged with a stirbar and 75-A (510 mg, 1.38 mmol). TFA (50%) in DCM (6 mL) is added and the solution is stirred for 1 h at room temperature. The solvent is removed in vacuo, hexanes is added and then evaporated again in vacuo to dryness, and repeated if necessary to azeotrope remaining TFA. The crude material is used directly in the next step without further purification.

75-C: Proline 75-B as the TFA salt, (1.07 g, 2.8 mmol) is dissolved in CH₂Cl₂ (30 mL); Reference Compound 4 (1.02 g, 2.7 mmol) and HATU (1.12 g, 2.94 mmol) are added, and the solution is stirred at room temp for 10 min DIEA (1.5 mL, 8.4 mmol) is added via syringe and the reaction mixture is allowed to stir overnight at room temperature. The solvent is removed in vacuo, and the crude material is directly purified by flash chromatography (120 g silica, hexanes/EtOAc gradient). The solvent is removed in vacuo to afford 75-C as an oily semisolid.

75-D: Methyl ester 75-C (1.15 g, 1.87 mmol) is dissolved in dioxane (15 mL). Lithium hydroxide (120 mg, 2.8 mmol) is dissolved in water (15 mL) and added dropwise to the solution of methyl ester 75-C, and allowed to stir for 3 h at room temperature. The reaction mixture is concentrated in vacuo to remove dioxane, acidified with 1M NaHSO₄, and extracted with EtOAc. The combined organic layers are washed with brine and dried with MgSO₄. The solvent is removed in vacuo to afford carboxylic acid 75-D as a waxy solid.

75-E: Carboxylic acid 75-D (102 mg, 0.17 mmol) is dissolved in DCM (5 mL). Reference Compound 13 (62 mg, 0.17 mmol) and HATU (71 mg, 0.19 mmol) are added, and the mixture is stirred for 10 min at room temperature. DIEA (0.10 mL, 0.51 mmol) is then added, and the reaction mixture is stirred overnight at room temperature. The solvent is removed in vacuo, the crude is redissolved in EtOAc (15 mL) and washed with 1M HCl (2×15 mL), followed by saturated aqueous NaHCO₃ (2×15 mL) and brine (15 mL), and dried with anhydrous Na₂SO₄. Solvent is removed and the residue is purified by flash chromatography (Hexanes/EtOAc) to afford the desired product as a white foam.

75-F: Alcohol 75-E (94 mg, 0.11 mmol) is dissolved in DCM (10 mL), and Dess-Martin periodinane (56 mg, 0.13 mmol) is added. The reaction mixture is stirred overnight at room temperature. The solvent is removed in vacuo and the crude is purified by flash chromatography using a gradient of EtOAc:Hexanes to afford the ketone as a white foam.

Example 75: Ketone 75-F (60 mg, 0.072 mmol) is dissolved in DCM (1 mL) and TFA 50% in DCM (5 mL) is added. The reaction is stirred at room temp for 2 h and the solvent is removed in vacuo. The crude material is purified by reverse-phase HPLC and the solvent is lyophilized to a white powder.

### EXAMPLES 76-91

Examples 76-91 are prepared following methods analogous to Examples 1 and 75, using appropriate acid and amine components that would be readily apparent to those skilled in the art.

Table 1 shows compounds of Formula (1), as described in Examples 1-91.

TABLE 1

| | | Physical Data |
|---|---|---|
| | | MS (m/z), Elemental |
| | | Analysis, and ¹H NMR |
| Compound | Structure | 400 MHz (DMSO-d₆) |
| 1 | 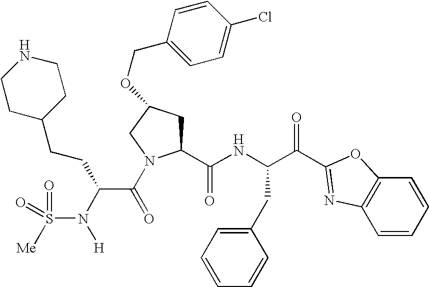 | MS m/z 750.3 (M + 1) |
| 2 | 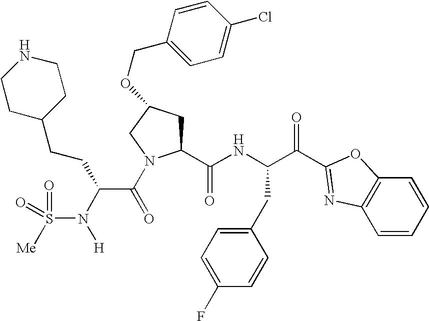 | MS m/z 768.3 (M + 1) |
| 3 | 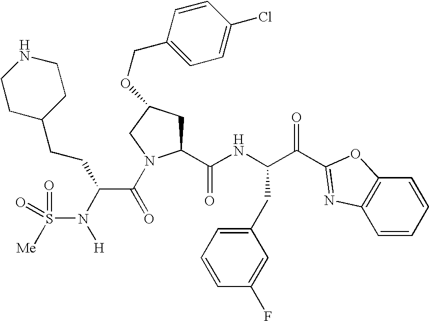 | MS m/z 768.3 (M + 1) |
| 4 | 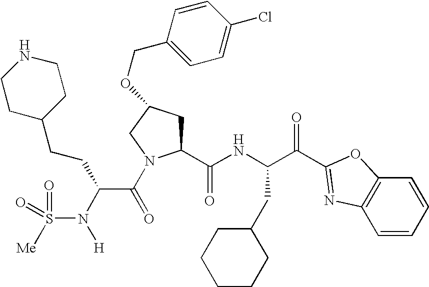 | MS m/z 756.4 (M + 1) |
| 5 | 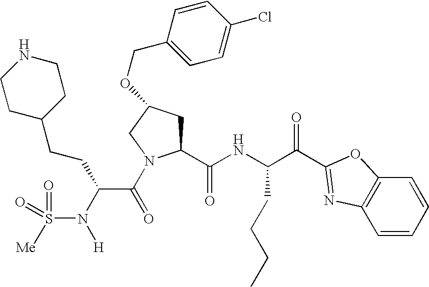 | MS m/z 715.3 (M + 1) |
| 6 | 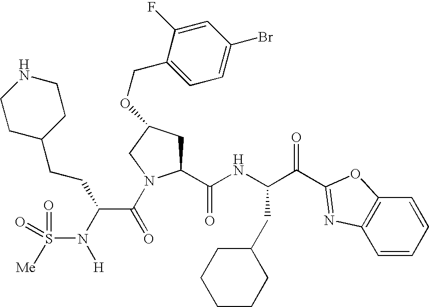 | MS m/z 818.3 and 820.3 (M + 1) |
| 7 | 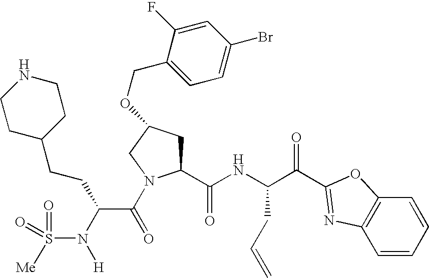 | MS m/z 762.2 and 764.2 (M + 1) |
| 8 | 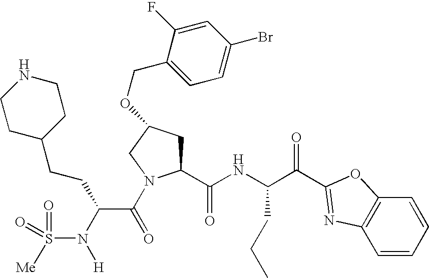 | MS m/z 764.3 and 766.3 (M + 1) |
| 9 | 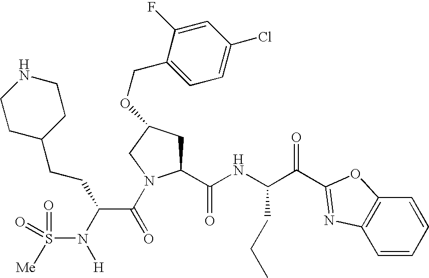 | MS m/z 720.3 (M + 1) |
| 10 | 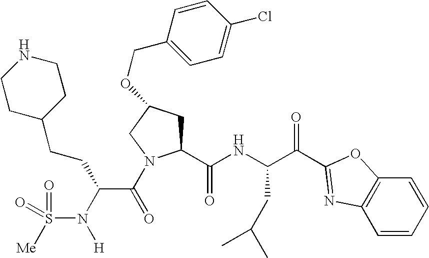 | MS m/z 716.3 (M + 1) |
| 11 | 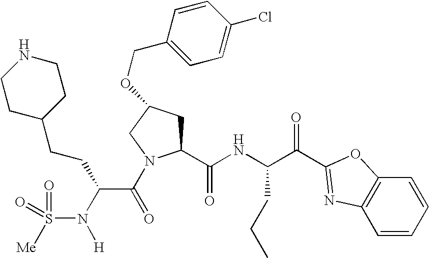 | MS m/z 702.3 (M + 1) |
| 12 | 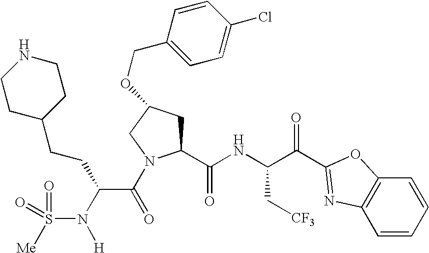 | MS m/z 742.3 (M + 1) |
| 13 | 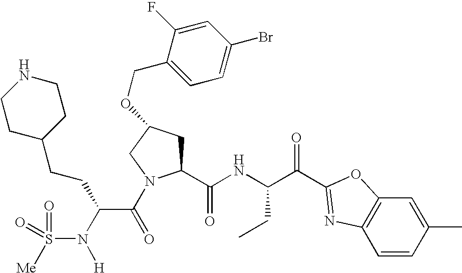 | MS m/z 764.3 and 766.3 (M + 1) |
| 14 | 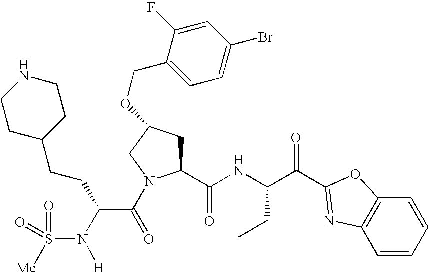 | MS m/z 750.2 and 752.2 (M + 1) |
| 15 | 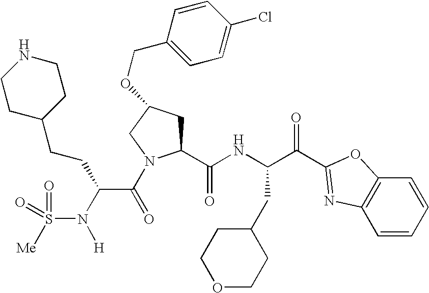 | MS m/z 758.4 (M + 1) |
| 16 | 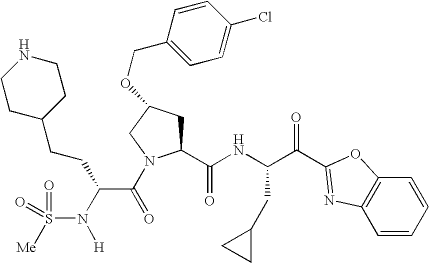 | MS m/z 714.3 (M + 1) |
| 17 | 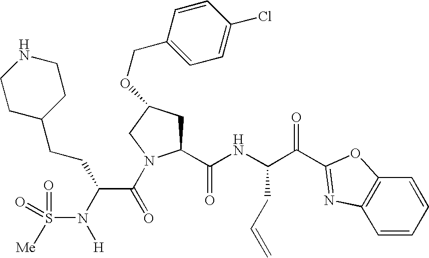 | MS m/z 700.3 (M + 1) |
| 18 | 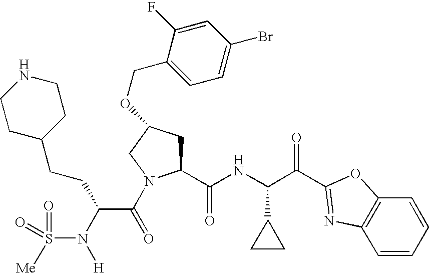 | MS m/z 762.2 and 764.2 (M + 1) |
| 19 | 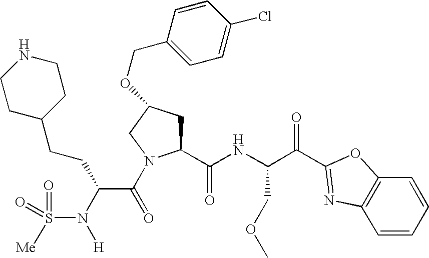 | MS m/z 704.3 (M + 1) |
| 20 | 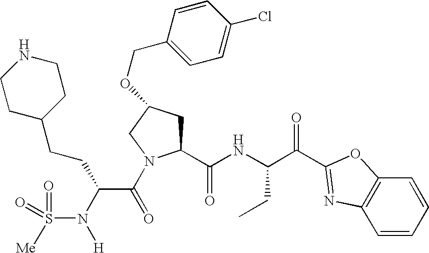 | MS m/z 702.3 (M + 1) |
| 21 | 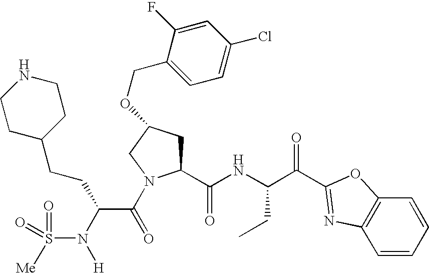 | MS m/z 706.3 (M + 1) |
| 22 | 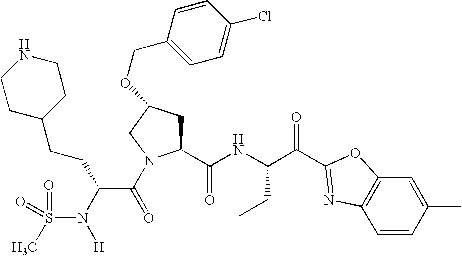 | MS m/z 702.3 (M + 1) |
| 23 | 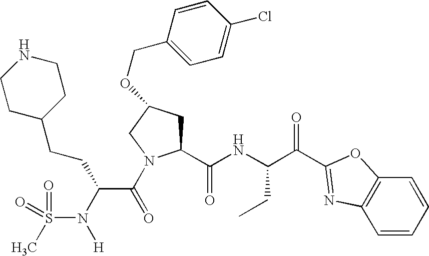 | MS m/z 688.3 (M + 1) |
| 24 | 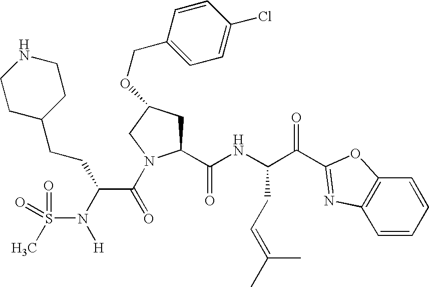 | MS m/z 727.4 (M + 1) |
| 25 | 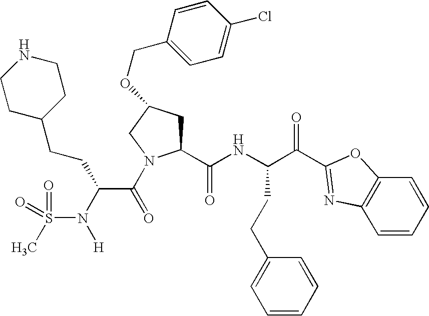 | MS m/z 764.4 (M + 1) |
| 26 | 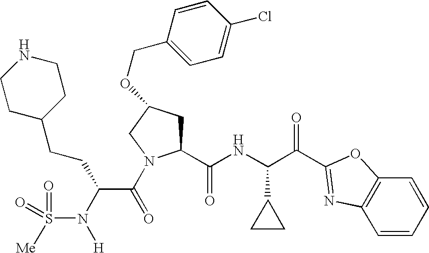 | MS m/z 670.3 (M + 1) |
| 27 | 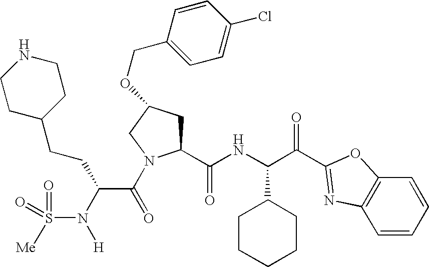 | MS m/z 742.4 (M + 1) |
| 28 | 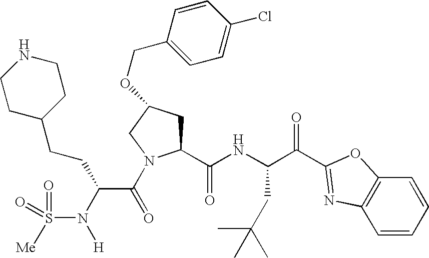 | MS m/z 730.4 (M + 1) |
| 29 | 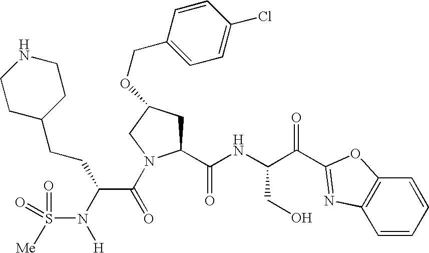 | MS m/z 690.3 (M + 1) |
| 30 | 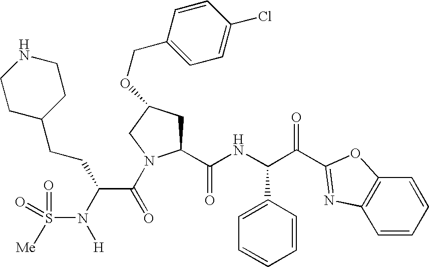 | MS m/z 736.3 (M + 1) |
| 31 | 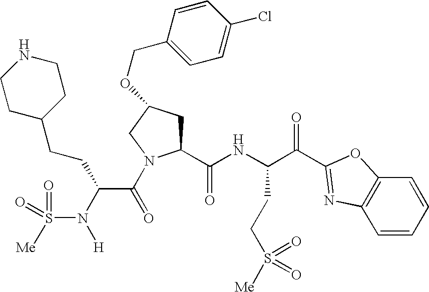 | MS m/z 766.3 (M + 1) |
| 32 | 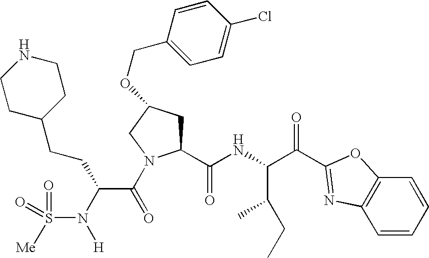 | MS m/z 716.3 (M + 1) |
| 33 | 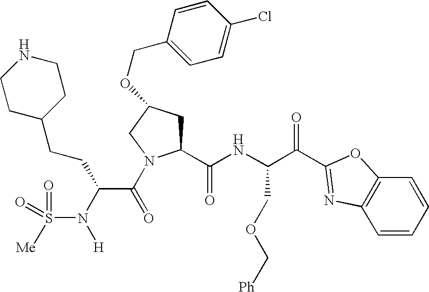 | MS m/z 780.3 (M + 1) |
| 34 | 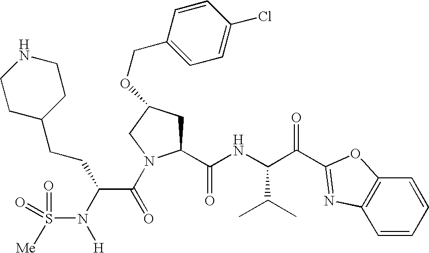 | MS m/z 702.3 (M + 1) |
| 35 | 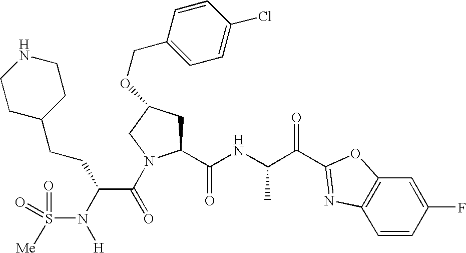 | MS m/z 692.3 (M + 1) |
| 36 | 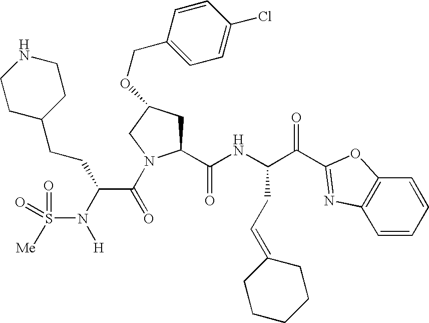 | MS m/z 767.4 (M + 1) |
| 37 | 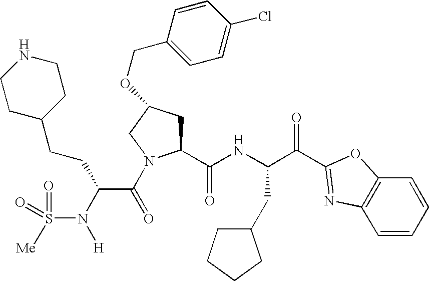 | MS m/z 742.4 (M + 1) |
| 38 | 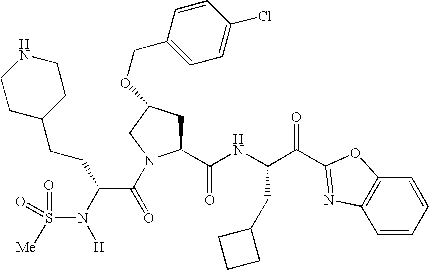 | MS m/z 728.3 (M + 1) |
| 39 | 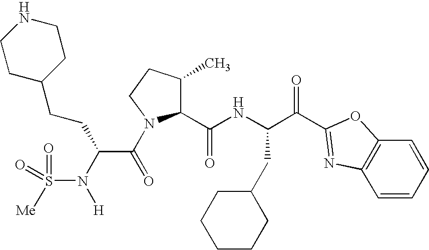 | MS m/z 716.4 (M + 1) |
| 40 | 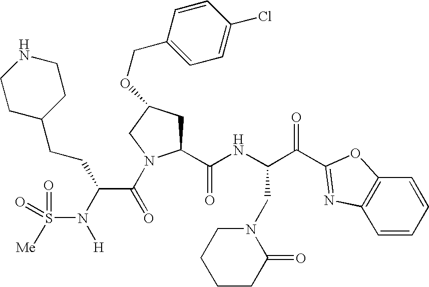 | MS m/z 671.3 (M + 1) |
| 41 | 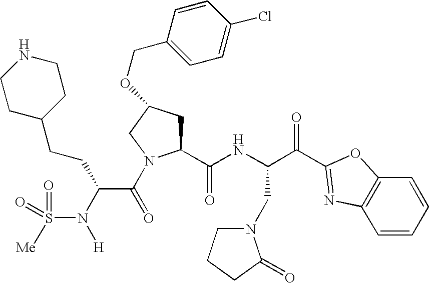 | MS m/z 657.3 (M + 1) |
| 42 | 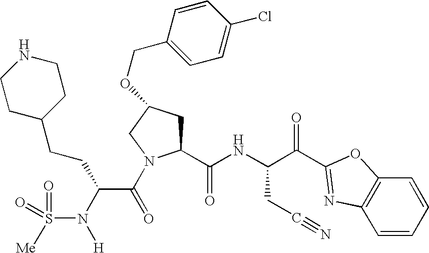 | MS m/z 699.3 (M + 1) |
| 43 | 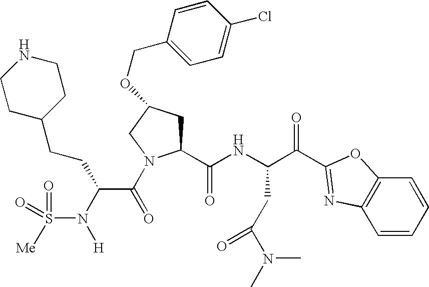 | MS m/z 745.3 (M + 1) |
| 44 | 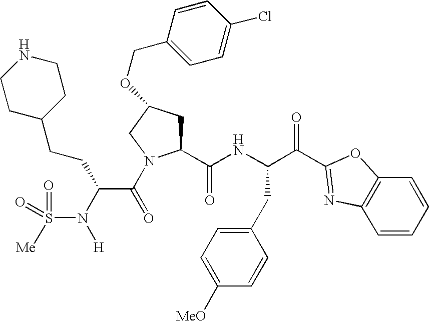 | MS m/z 780.3 (M + 1) |
| 45 | 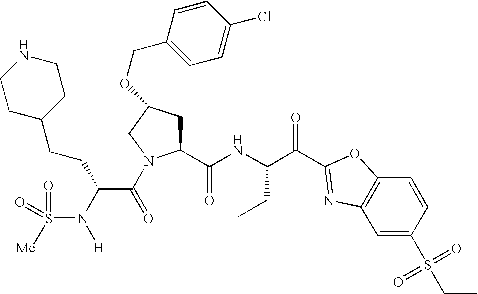 | MS m/z 780.3 (M + 1) |
| 46 | 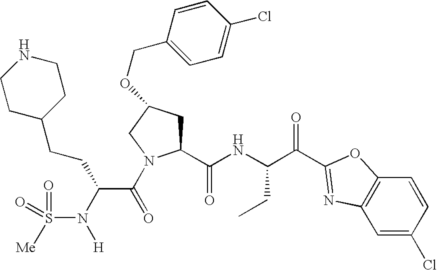 | MS m/z 722.3 (M + 1) |
| 47 | 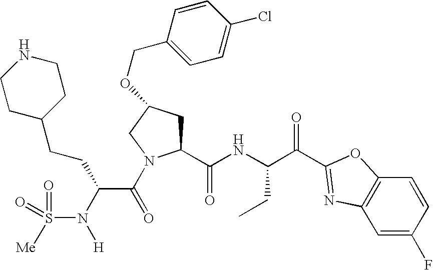 | MS m/z 706.3 (M + 1) |
| 48 | 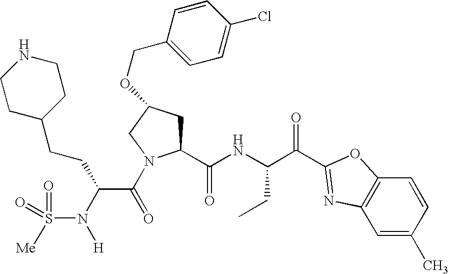 | MS m/z 702.3 (M + 1) |
| 49 | 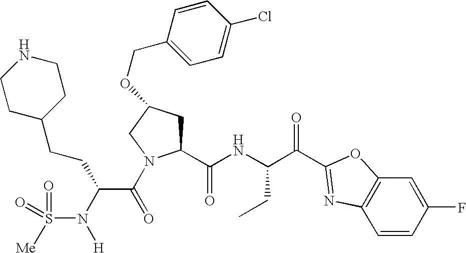 | MS m/z 706.3 (M + 1) |
| 50 | 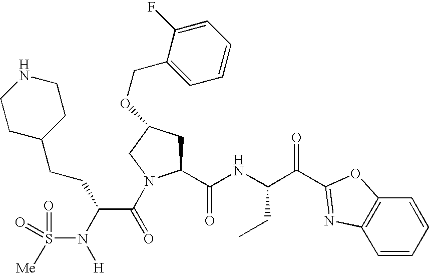 | MS m/z 672.3 (M + 1) |
| 51 | 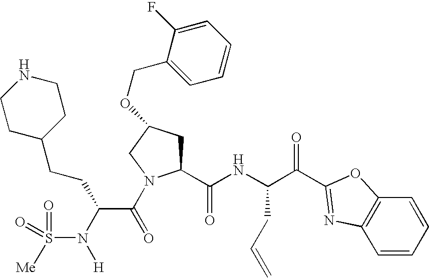 | MS m/z 684.3 (M + 1) |
| 52 | 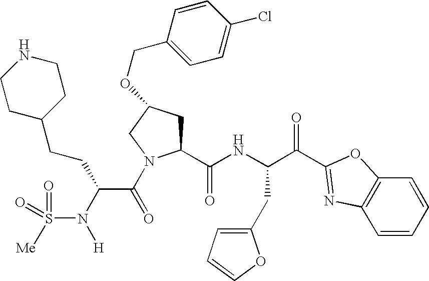 | MS m/z 740.3 (M + 1) |
| 53 | 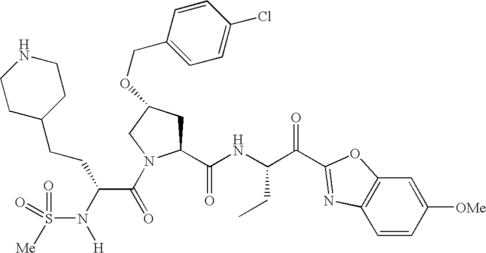 | MS m/z 718.3 (M + 1) |
| 54 | 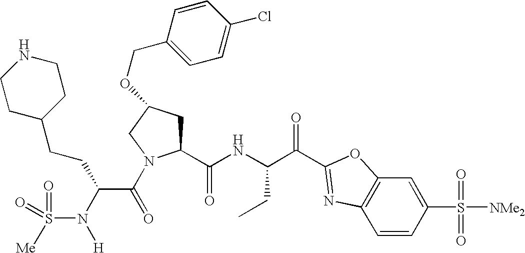 | MS m/z 795.3 (M + 1) |
| 55 | 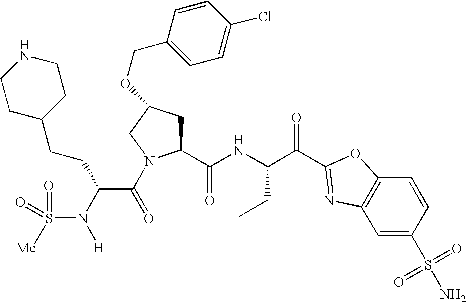 | MS m/z 767.3 (M + 1) |
| 56 | 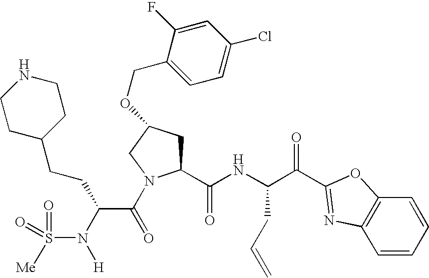 | MS m/z 718.3 (M + 1) |
| 57 | 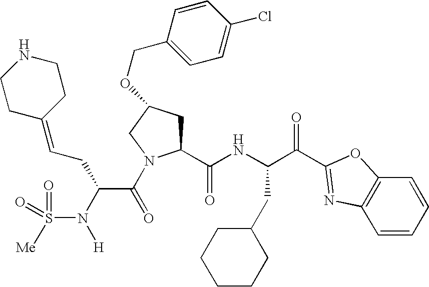 | MS m/z 754.4 (M + 1) |
| 58 | 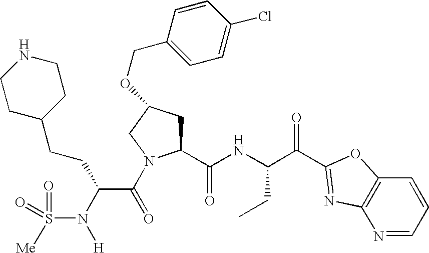 | MS m/z 689.3 (M + 1) |
| 59 | 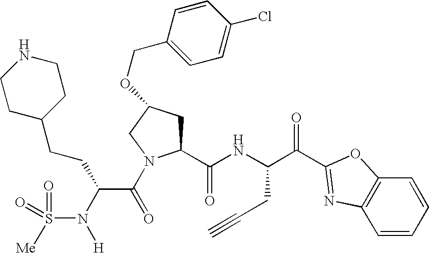 | MS m/z 698.3 (M + 1) |
| 60 | 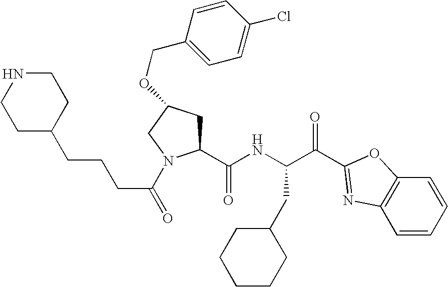 | MS m/z 732.3 (M + 1) |
| 61 | 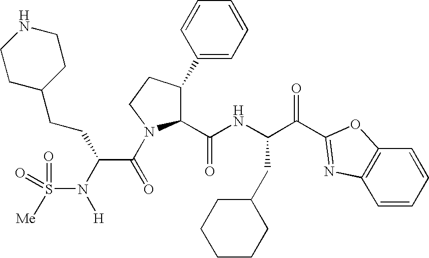 | MS m/z 691.5 (M + 1) |
| 62 | 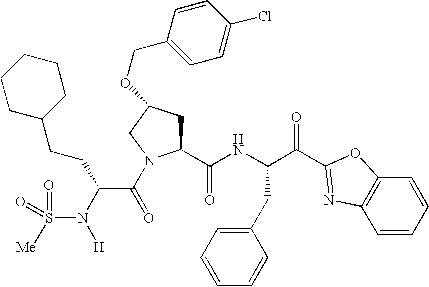 | MS m/z 749.3 (M + 1) |
| 63 | 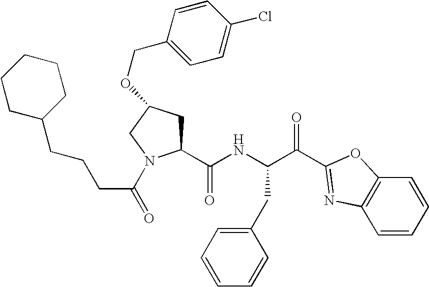 | MS m/z 657.3 (M +1) |
| 64 | 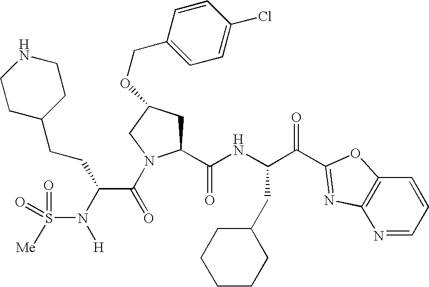 | MS m/z 757.4 (M + 1) |
| 65 | 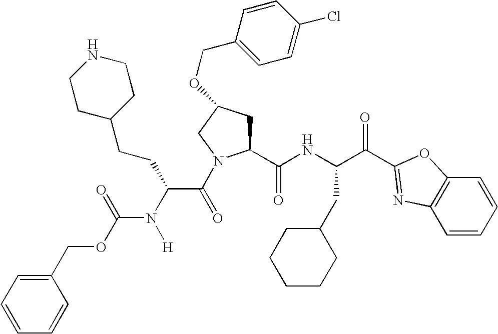 | MS m/z 813.4 (M + 1) |
| 66 | 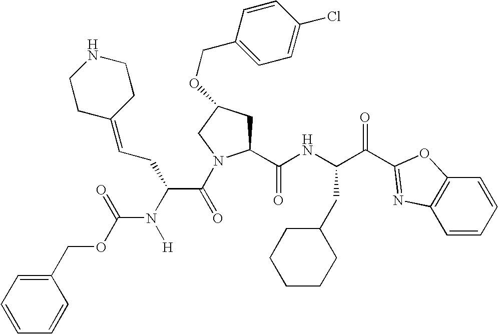 | MS m/z 811.4 (M + 1) |
| 67 | 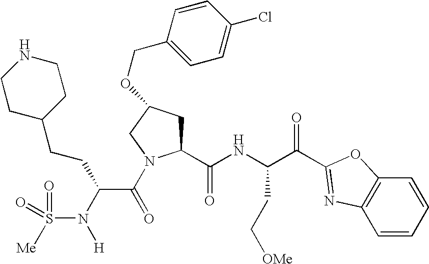 | MS m/z 718.3 (M + 1) |
| 68 | 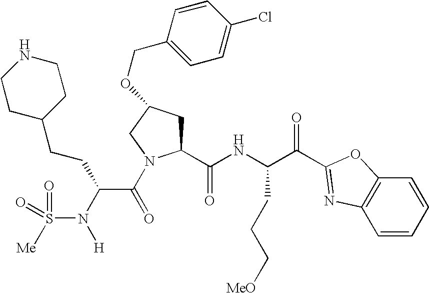 | MS m/z 732.3 (M + 1) |
| 69 | 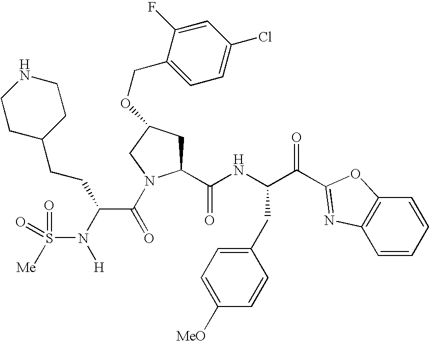 | MS m/z 798.3 (M + 1) |
| 70 | 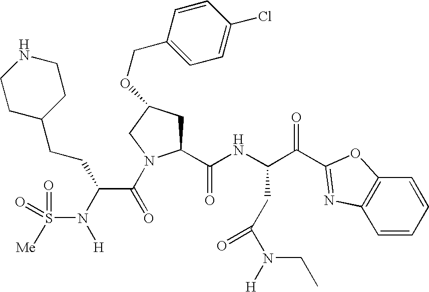 | MS m/z 745.3 (M + 1) |
| 71 | 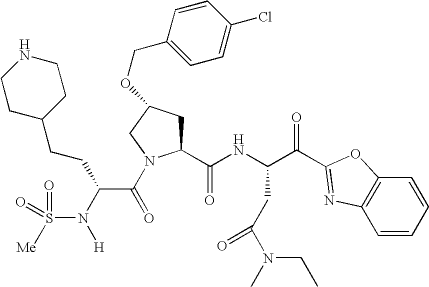 | MS m/z 759.3 (M + 1) |
| 72 | 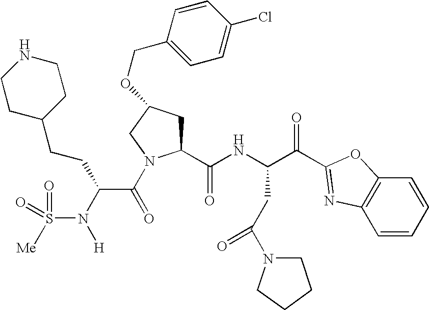 | MS m/z 771.3 (M + 1) |
| 73 | 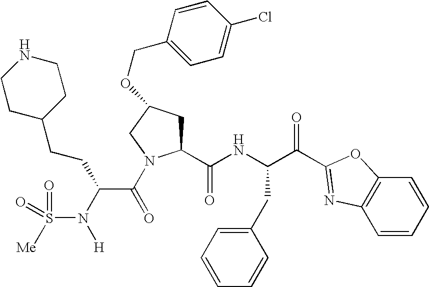 | MS m/z 743.3 (M + 1) |
| 74 | 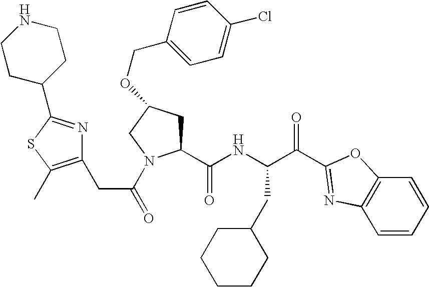 | MS m/z 732.3 (M + 1) |
| 75 | 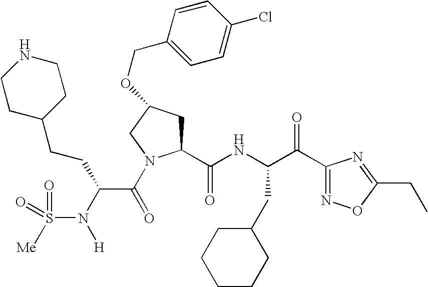 | MS m/z 735.4 (M + 1) |
| 76 | 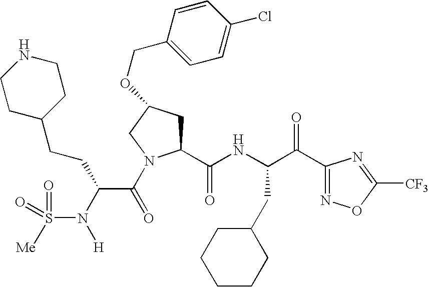 | MS m/z 775.3 (M + 1) |
| 77 | 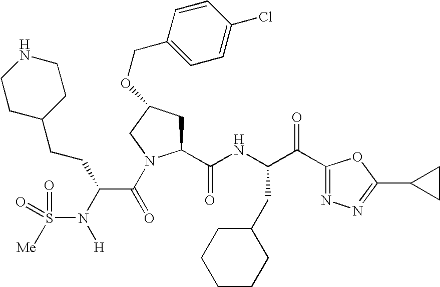 | MS m/z 747.4 (M + 1) |
| 78 | 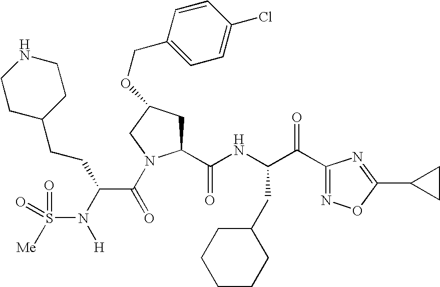 | MS m/z 747.4 (M + 1) |
| 79 | 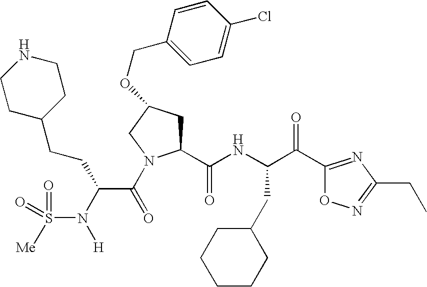 | MS m/z 735.4 (M + 1) |
| 80 | 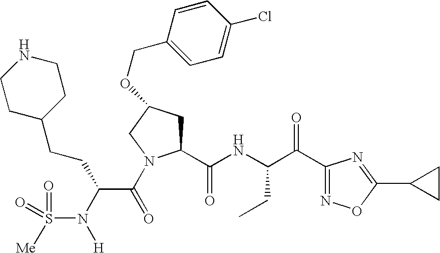 | MS m/z 679.3 (M + 1) |
| 81 | 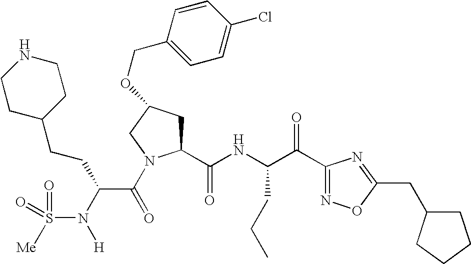 | MS m/z 735.3 (M + 1) |
| 82 | 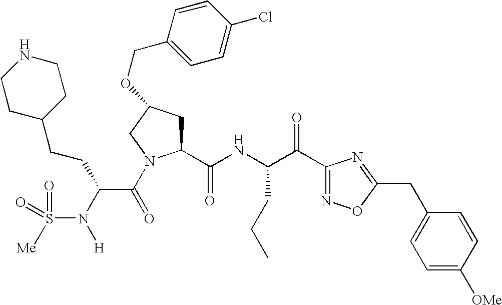 | MS m/z 773.3 (M + 1) |
| 83 | 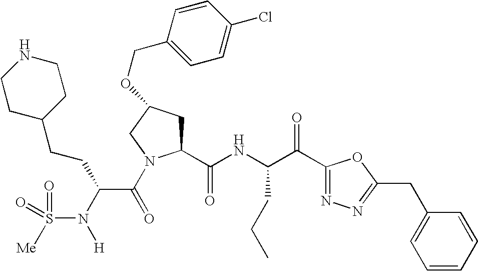 | MS m/z 743.3 (M + 1) |
| 84 | 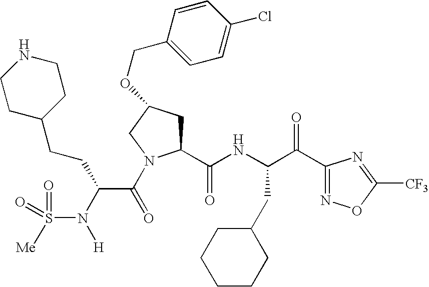 | MS m/z 775.3 (M + 1) |
| 85 | 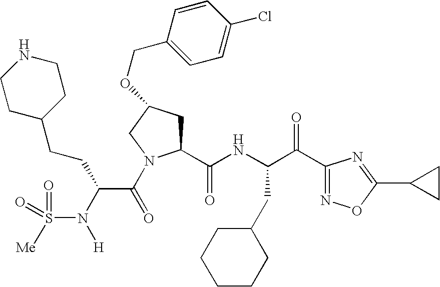 | MS m/z 747.3 (M + 1) |
| 86 | 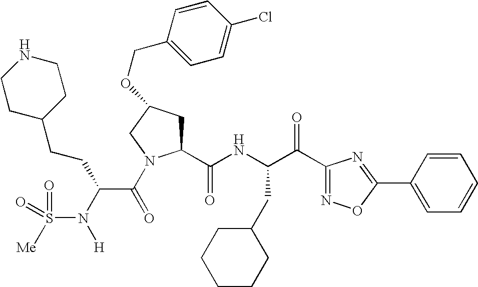 | MS m/z 783.3 (M + 1) |
| 87 | 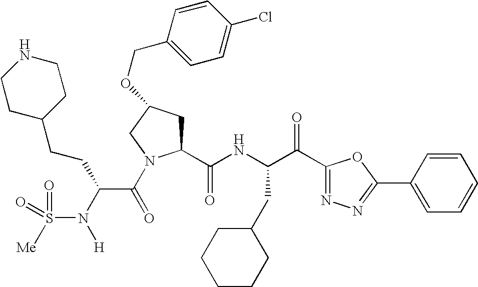 | MS m/z 783.3 (M + 1) |
| 88 | 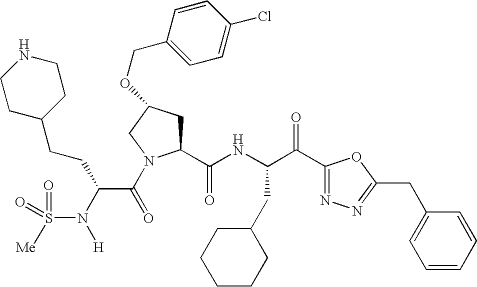 | MS m/z 797.4 (M + 1) |
| 89 | 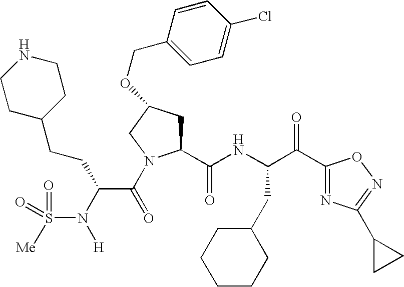 | MS m/z 747.3 (M + 1) |
| 90 | 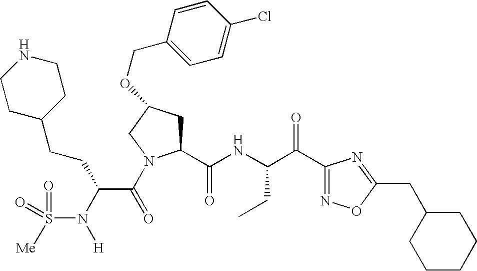 | MS m/z 735.3 (M + 1) |
| 91 | 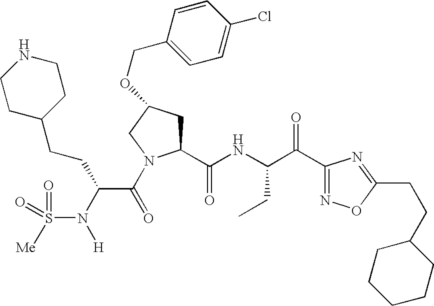 | MS m/z 749.4 (M + 1) |

Assays

The suitability of a channel activating protease inhibitor such as a prostasin inhibitor for the treatment of a disease mediated by inhibition of a channel activating protease may be tested by determining the inhibitory effect of the channel activating protease inhibitor on: (1) the native, isolated, purified or recombinant channel activating protease, using a suitable biochemical assay format, using the method described in Shipway et al.; Biochemical and Biophysical Research Communications 2004; 324(2):953-63; and/or (2) the ion channel/ion transport function in suitable isolated cells or confluent epithelia, using the methods described in Bridges et al.; American Journal of Physiology Lung Cell Molecular Physiology 2001; 281(1):L16-23; and Donaldson et al.; Journal of Biological Chemistry 2002; 277(10):8338-45.

Biochemical Assays

Recombinant human prostasin and matriptase and guinea pig prostasin are generated according to methods described in Shipway et al., Biochem. and Biophys. Res. Commun. 2004; 324(2):953-63. The recombinant enzymes are incubated in an electrolyte buffer containing the test compounds or vehicle in a suitable multiple well assay plate such as a 96 or 384 well plate. At a defined time after the mixing of enzyme with compound or vehicle, a suitable fluorescent peptide substrate is added to the assay mixture. As substrate becomes cleaved by the active enzyme, fluorescence (measured, using a suitable fluorescence plate reader) increases and the rate of turnover of substrate (i.e. enzyme activity) may be quantified and thus the inhibitory effect of any test compound. The efficacy of test compounds is expressed as the concentration that induces 50% attenuation in the enzyme activity (Kᵢ).

In general, compounds of the invention may have Kᵢ values from 0.1 nM to 5 μM. In some examples, compounds of the invention may have Kᵢ values from 0.1 nM to 500 nM; from 0.1 nM to 50 nM; from 0.1 nM to 5 nM; or from 0.1 nM to 0.5 nM. In particular examples, compounds of the invention may have Kᵢ values from 0.1 nM to 0.5 nM; from 0.5 nM to 5 nM; from 5 nM to 50 nM; from 50 nM to 500 nM; or from 500 nM to 5 μM. In yet other examples, compounds may have Kᵢ values less than 0.1 nM or more than 5 μM.

Epithelial Ion Transport

Human bronchial epithelial cells are cultured according to methods described in Danahay et al., Am. J. Physiol. Lung Cell Mol. Physiol. 2002; 282(2):L226-36. When suitably differentiated (days 14-21 after establishing an apical-air interface), epithelial cells are treated with either vehicle, aprotinin (200 μg/ml) or test compound for 90 minutes. Epithelia are then placed into chambers as described in Danahay et al., supra, maintaining the concentration of vehicle, aprotinin or test compound on the apical side of the epithelia. Short circuit current (ISC) is then measured by voltage clamping the epithelia to zero millivolts. The amiloride-sensitive ISC is then measured by the addition of amiloride (10 μM) to the apical surface of the epithelia. The potency of the test compound is expressed as the concentration inducing a 50% inhibition of the total aprotinin-sensitive component of the amiloride-sensitive ISC.

In general, compounds of the invention may have IC₅₀ values from 1 nM to 10 μM. In some examples, compounds of the invention may have IC₅₀ values from 1 nM to 1 μM; or more particularly from 1 nM to 100 nM. In yet other examples, compounds of the invention may have IC₅₀ values from 100 nM to 1 μM, or from 1 μM to 10 μM. In yet other examples, compounds may have IC₅₀ values less than 1 nM or more than 10 μM.

Tracheal Potential Difference (In Vivo)

Guinea pigs are anaesthetized, using a short acting inhalation anaesthesia such as halothane and N₂0. While under short acting anaesthesia, an oral gavage needle is inserted into the trachea via the oropharangeal route. Once inside the trachea, a small volume (50-200 μl) of vehicle or test compound, in a suitable aqueous-based diluent, is instilled into the airways. Animals then recover and become fully ambulatory. Alternatively, test compounds may be administered to animals, using aerosol or dry powder dosing. At a defined time after dosing, the animals are surgically anaesthetized, using a suitable anaesthesia such as ketamine and xylazine. The trachea is then exposed and a plastic agar bridge electrode is inserted into the tracheal lumen. A reference electrode is also inserted into the layers of muscle in the animal's neck. The tracheal potential difference is then measured, using a suitable high impedance voltmeter as described in Takahashi et al., Toxicol Appl Pharmacol. 1995; 131(1):31-6. The potency of the test compound is expressed as the dose inducing a 50% reduction in the sensitive-component of the tracheal potential difference.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A compound of Formula (1): 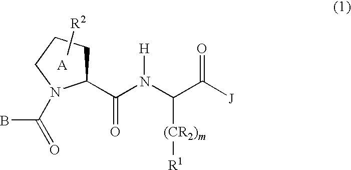
or pharmaceutically acceptable salts thereof; wherein
B is 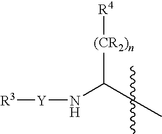
or (CR₂)ₖ—R⁵;
Y is —SO₂—, —NHCO—, —CO— or —O—C(═O)—, provided Y is SO₂ when R² is C₁₋₆ alkyl or phenyl;
J is an optionally substituted 5-12 membered monocyclic or fused heterocyclic ring comprising one or more heteroatoms selected from N, O, and S;
R¹ is H, an optionally halogenated C₁₋₆ alkyl, C₂₋₆ alkenyl or C₃₋₆ alkynyl; cyano, OH, O(CR₂)₁R⁶, SO₂R⁶, CONR(CR₂)₁R⁶, CONR⁷R⁸ or 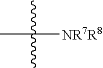
wherein R⁷ and R⁸ together with N in NR⁷R⁸ form an optionally substituted 5-7 membered heterocyclic ring attached to (CR₂)ₘ via a nitrogen atom; or R¹ is an optionally substituted C₃₋₇ cycloalkyl, aryl, or a 5-7 membered heterocyclic ring or heteroaryl having no nitrogen atoms; or 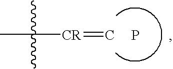
wherein ring P is an optionally substituted 5-7 membered carbocyclic ring;
R² is C₁₋₆ alkyl, C₂₋₆ alkenyl, C₂₋₆ alkynyl, aryl or -L-(CR₂)ₚ—R⁵ wherein L is O, S, S(O), SO₂ or OC(O);
R³ is C₁₋₆ alkyl, C₂₋₆ alkenyl, C₂₋₆ alkynyl or —(CR₂)₁—R⁵;
R⁴ is H, C₁₋₆ alkyl, C₂₋₆ alkenyl, —CR═CR—R⁶, C₂₋₆ alkynyl, or an optionally substituted 5-12 membered carbocyclic ring, heterocyclic ring, aryl or heteroaryl; or R⁴ is 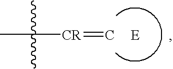
wherein ring E is an optionally substituted 5-12 membered monocyclic or fused carbocyclic or heterocyclic ring;
R⁵ and R⁶ are independently an optionally substituted 5-12 membered carbocyclic ring, heterocyclic ring, aryl or heteroaryl; or R⁶ may be C₁₋₆ alkyl or C₂₋₆ alkenyl;
each R is H, or C₁₋₆ alkyl, C₂₋₆ alkenyl, or C₂₋₆ alkynyl;
1 is 0-6; and
k, m, n, and p are independently 1-6.

2. The compound of claim 1, wherein J is benzoxazolyl; 1,2,3-oxadiazol-4-yl; 1,3,4-oxadiazol-2-yl; 1,2,4-oxadiazol-3-yl; oxazolo[4,5-b]pyridin-2-yl, oxazolo[4,5-c]pyridin-2-yl, oxazolo[5,4-c]pyridin-2-yl or oxazolo[5,4-b]pyridin-2-yl, each of which is optionally substituted with C₁₋₆ alkyl, halo, cyclopropyl, SO₂(C₁₋₆alkyl), OCH₃, SO₂N(CH₃)₂, SO₂NH₂, CF₃ or —(CR₂)₁—R⁵.

3. The compound of claim 1, wherein J is 1,2,4-oxadiazol-3-yl, and is optionally substituted with C₁₋₆ alkyl, CF₃ or —(CR₂)₀₋₁—R⁵ wherein R⁵ is an optionally substituted phenyl or C₃₋₇ cycloalkyl.

4. The compound of claim 1, wherein R¹ is C₁₋₆ alkyl, C₂₋₆ alkenyl, C₂₋₆ alkynyl, CF₃, OH, C₁₋₆ alkoxy, O(benzyl), SO₂(C₁₋₆ alkyl), CONH(C₁₋₆ alkyl), CON(C₁₋₆alkyl)₂, or cyano; or R¹ is phenyl, cyclopropyl, cyclobutyl, cyclopentyl, cyclohexyl, tetrahydropyranyl, furanyl, piperidin-2-onyl, pyrrolidin-2-onyl, pyrrolidin-1-carbonyl, 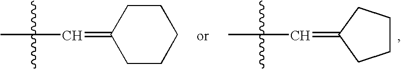
each of which is optionally substituted with halo, C₁₋₆ alkyl, C₂₋₆ alkenyl, C₃₋₆ alkynyl, cyano, OH or C₁₋₆ alkoxy.

5. The compound of claim 1, wherein R² is C₁₋₆ alkyl, an optionally substituted phenyl, or -L-(CR₂)ₚ—R⁵ wherein L is O.

6. The compound of claim 1, wherein said compound have Formula (2): 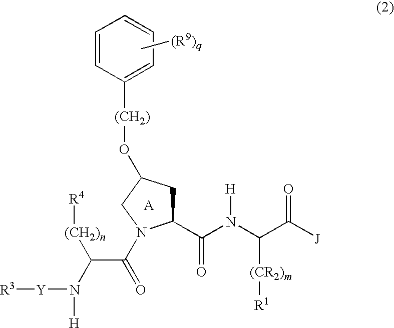
wherein J is benzoxazolyl; 1,2,3-oxadiazol-4-yl; 1,3,4-oxadiazol-2-yl; 1,2,4-oxadiazol-3-yl; oxazolo[4,5-b]pyridin-2-yl, oxazolo[4,5-c]pyridin-2-yl, oxazolo[5,4-c]pyridin-2-yl or oxazolo[5,4-b]pyridin-2-yl, each of which is optionally substituted with C₁₋₆ alkyl, halo, cyclopropyl, SO₂(C₁₋₆alkyl), OCH₃, SO₂N(CH₃)₂, SO₂NH₂, CF₃ or —(CR₂)₁—R⁵;
Y is SO₂ or —O—C(═O)—;
q is 1-5; and
R⁹ is halo, C₁₋₆ alkyl, or O(C₁₋₆ alkyl).

7. The compound of claim 6, wherein J is 1,2,4-oxadiazol-3-yl, and is optionally substituted with C₁₋₆ alkyl, CF₃ or —(CR₂)₀₋₁—R⁵ wherein R⁵ is an optionally substituted phenyl or C₃₋₇ cycloalkyl.

8. The compound of claim 6, wherein Y is SO₂ and R³ is C₁₋₆ alkyl.

9. The compound of claim 6, wherein q is 1-2 and R⁹ is halo.

10. The compound of claim 6, wherein R⁴ is an optionally substituted piperidinyl, cyclohexyl, phenyl,

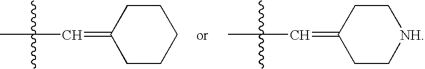

11. The compound of claim 10, wherein R⁴ is piperidinyl.

12. The compound of claim 1, wherein said compound has Formula (3): 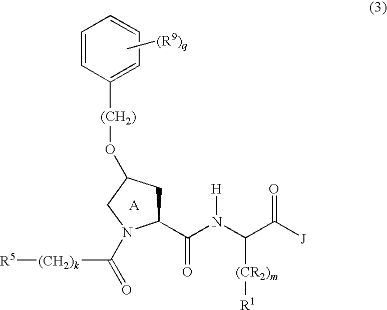
wherein R¹ is C₃₋₇ cycloalkyl or phenyl;
q is 1-5; and
R⁹ is halo, C₁₋₆ alkyl, or O(C₁₋₆ alkyl).

13. The compound of claim 11, wherein R⁵ is an optionally substituted cyclohexyl, piperidinyl or a thiazolyl.

14. The compound of claim 1, selected from the group consisting of:

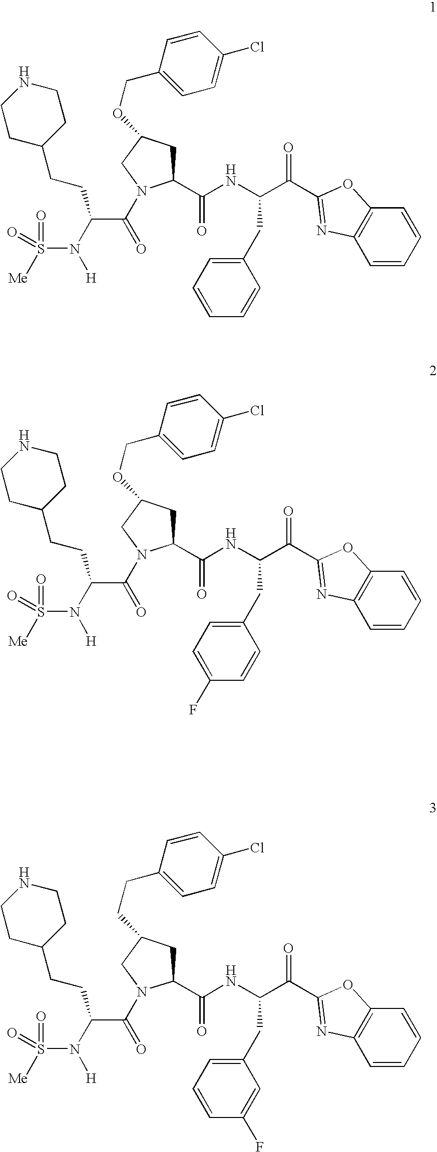

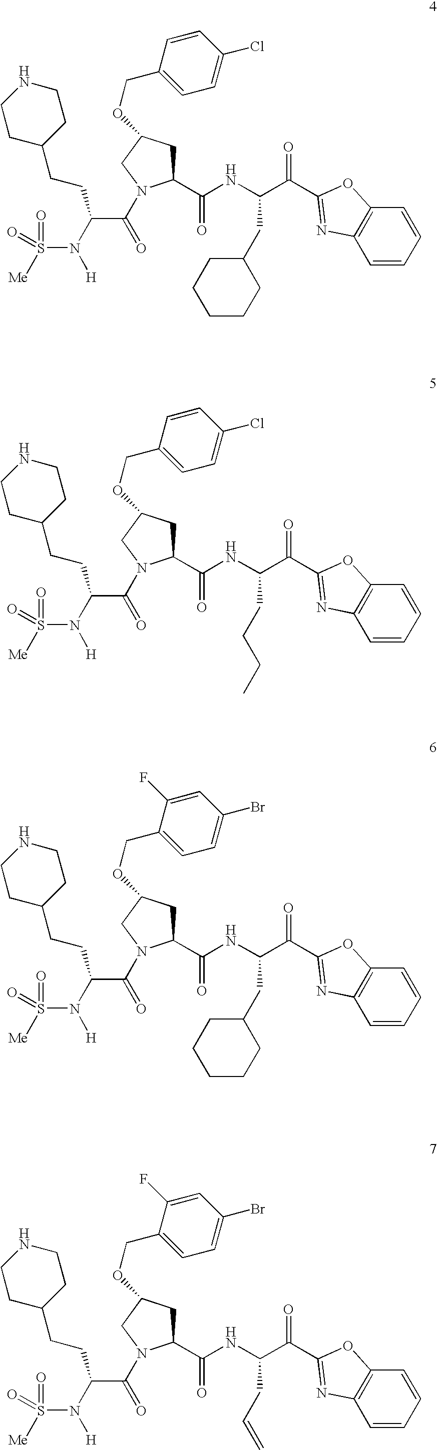

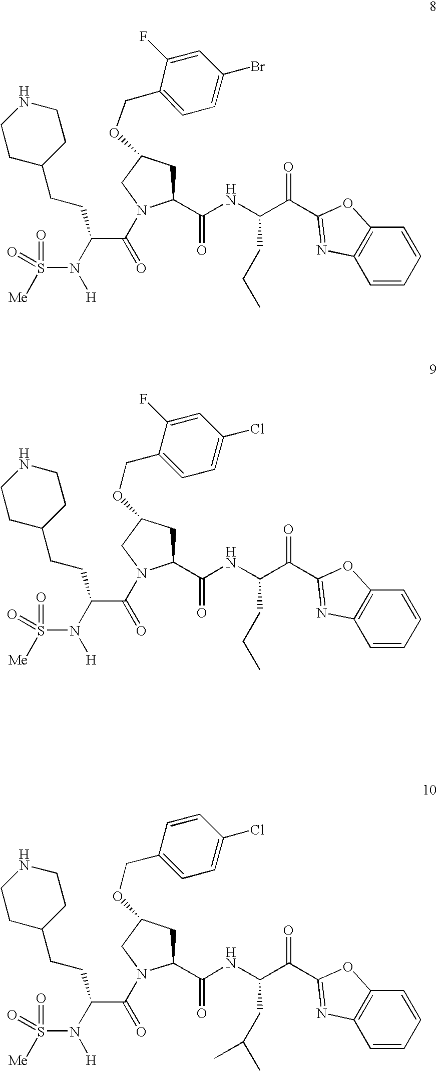

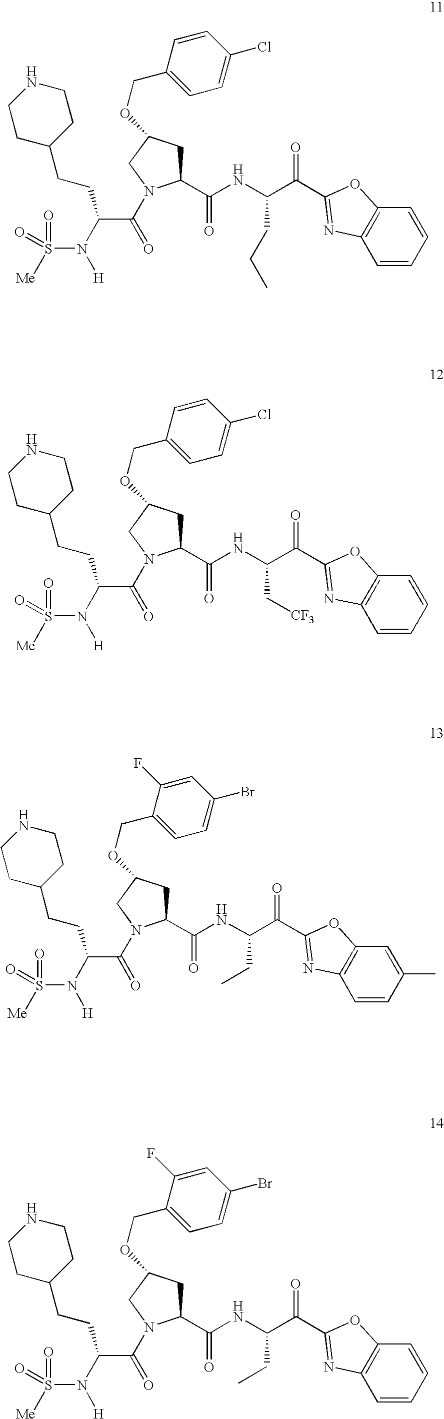

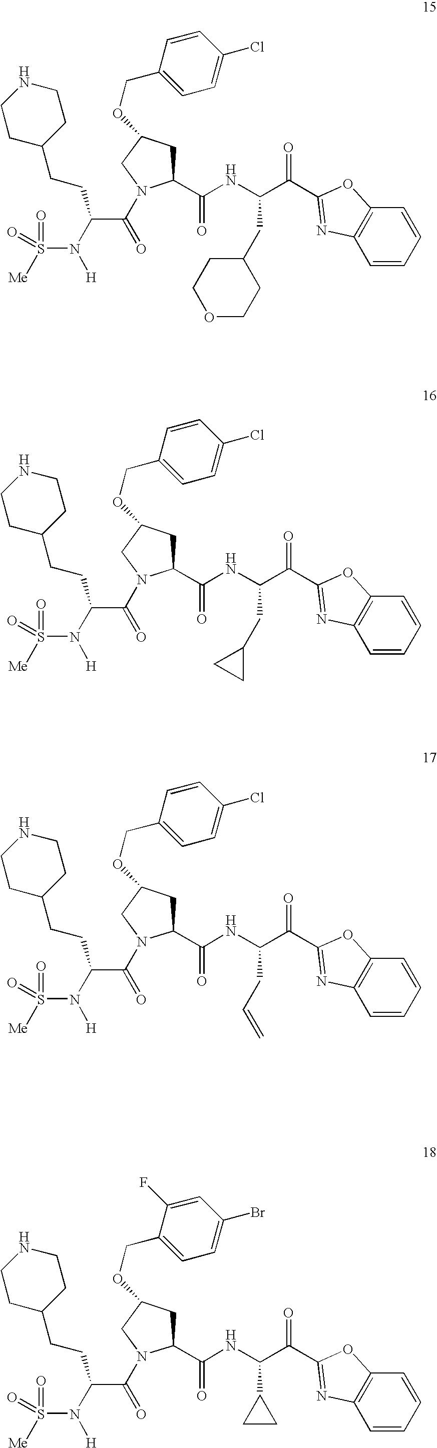

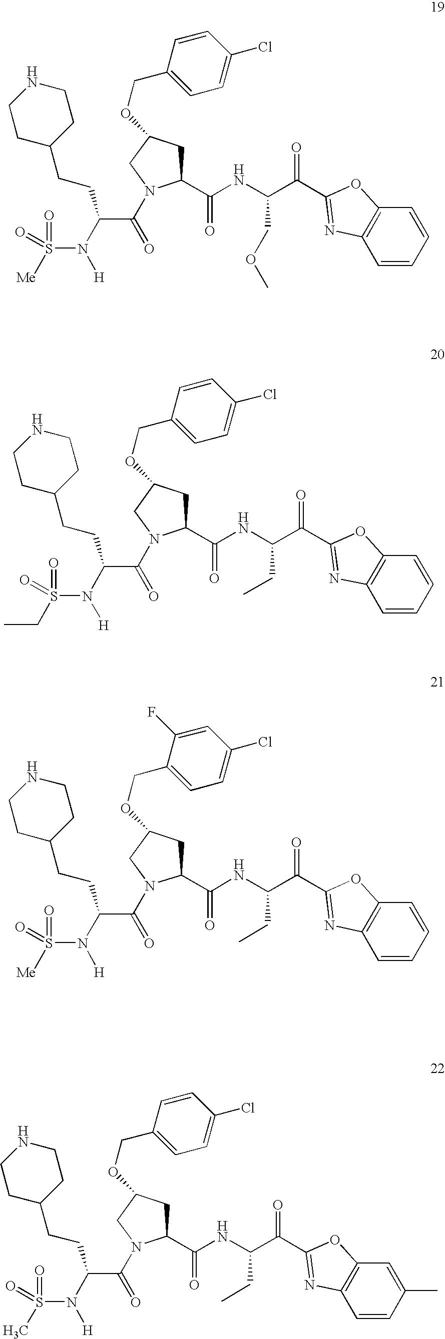

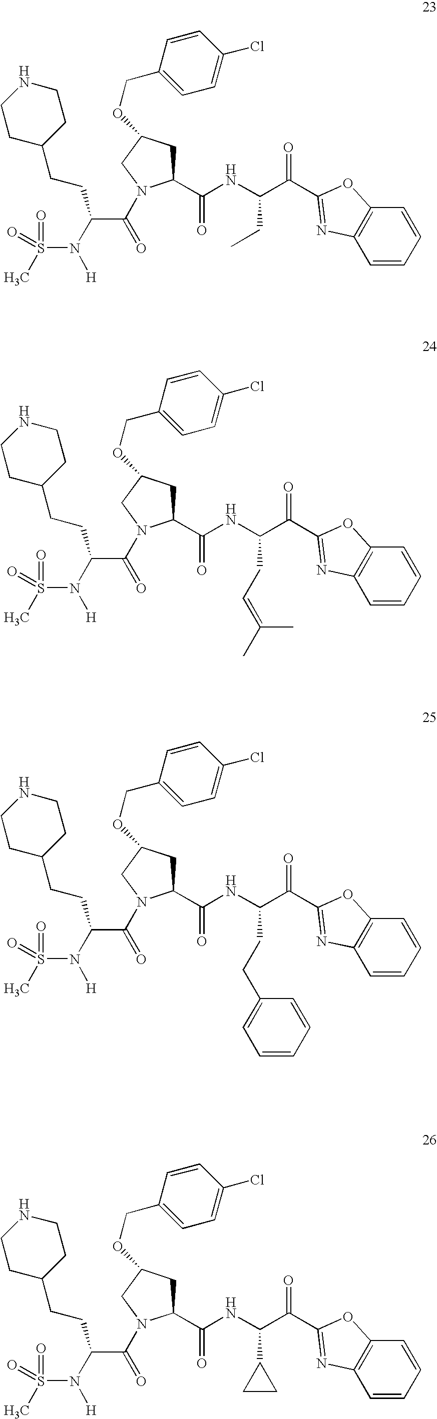

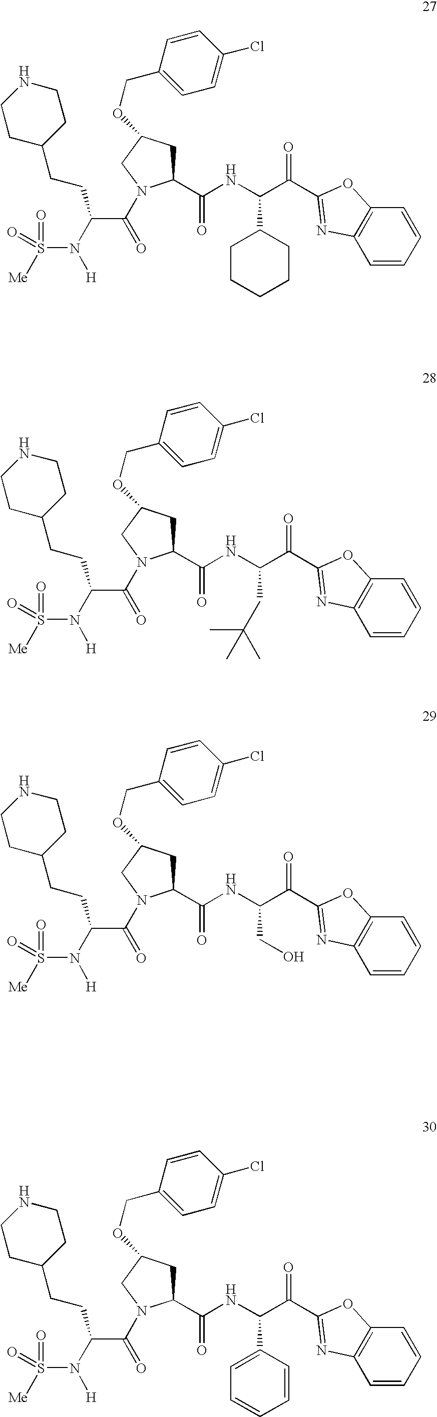

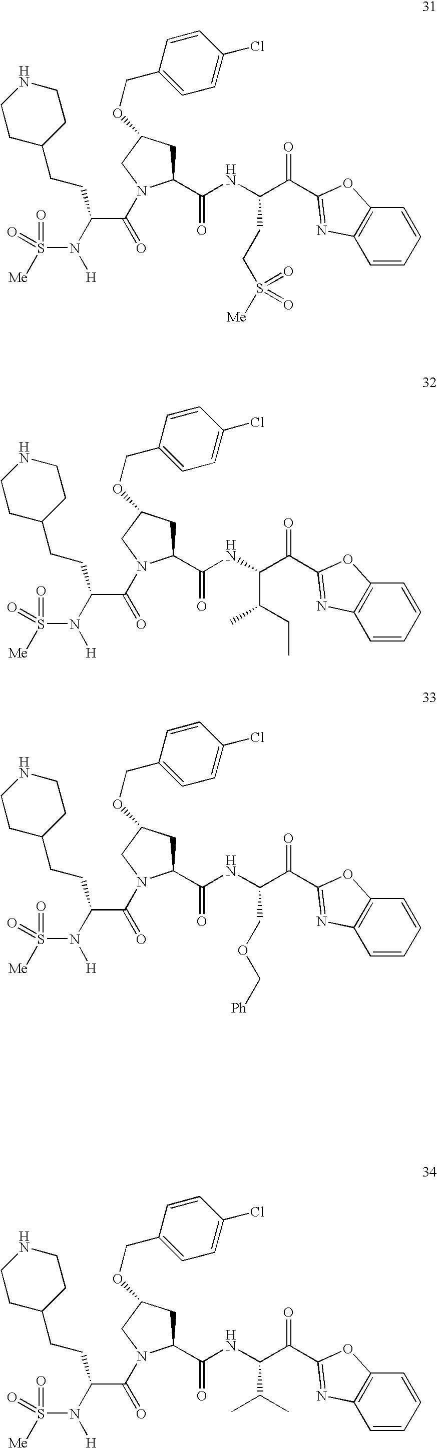

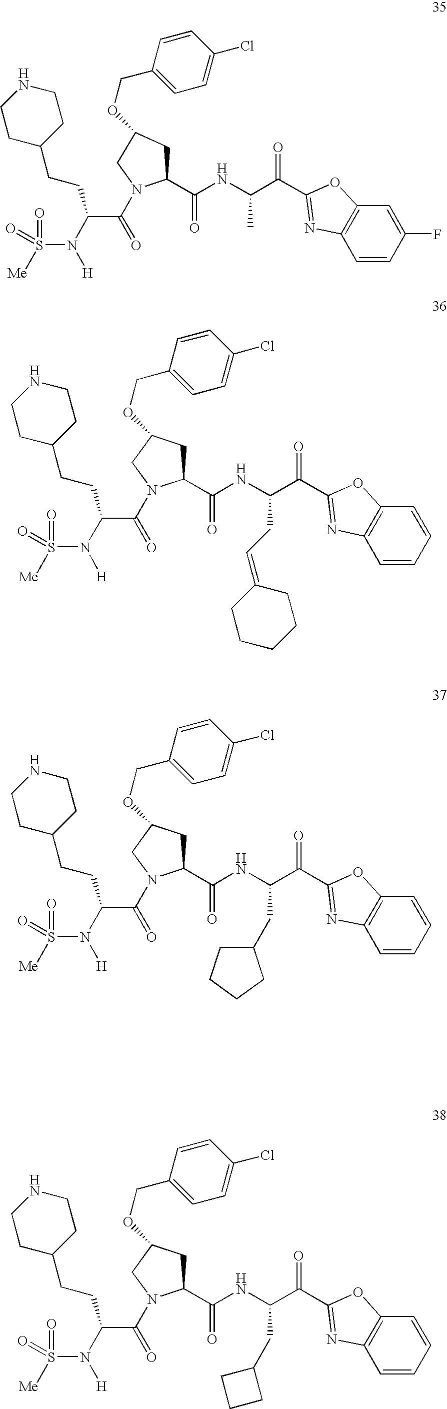

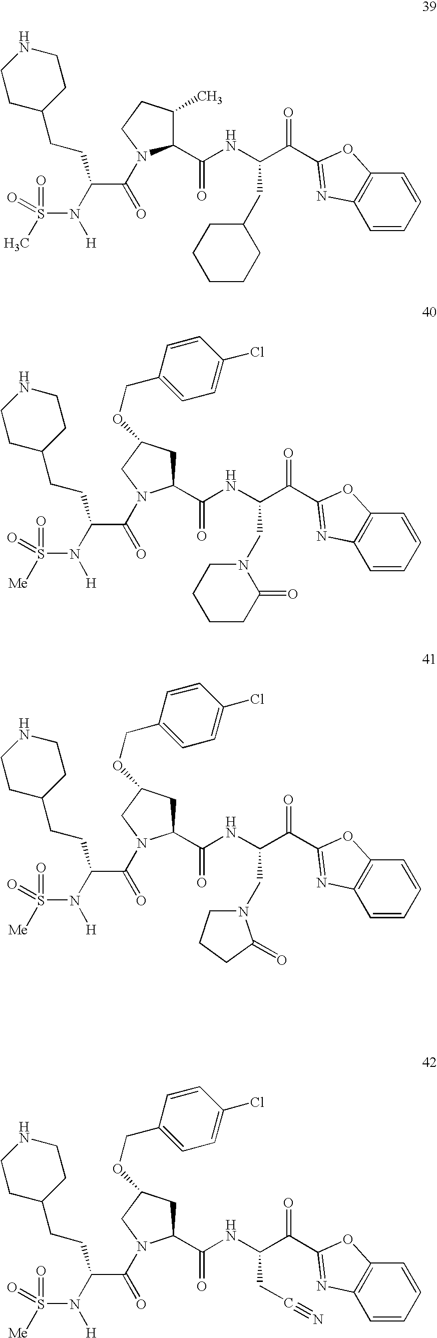

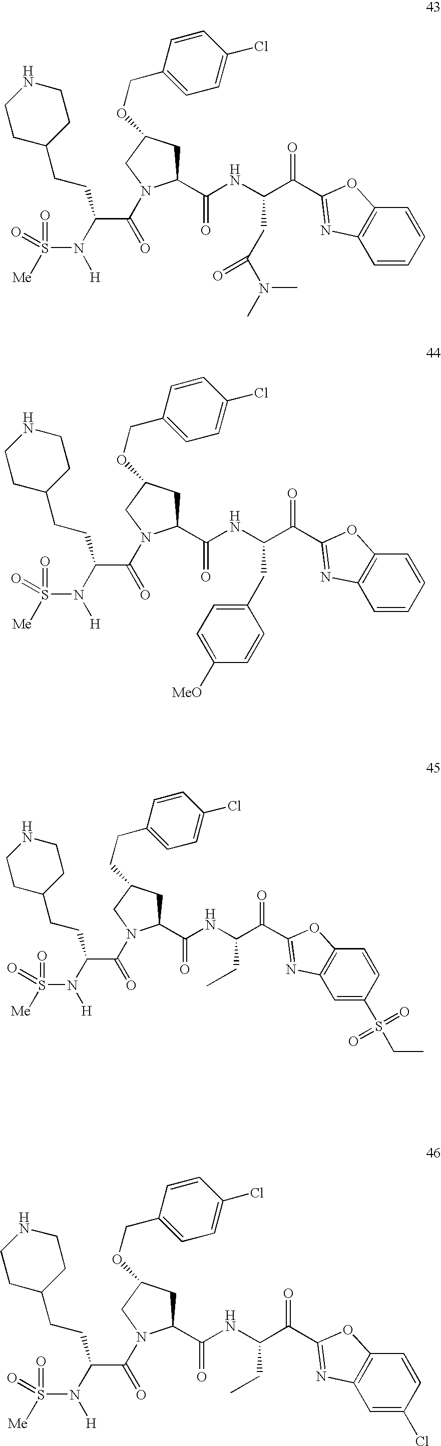

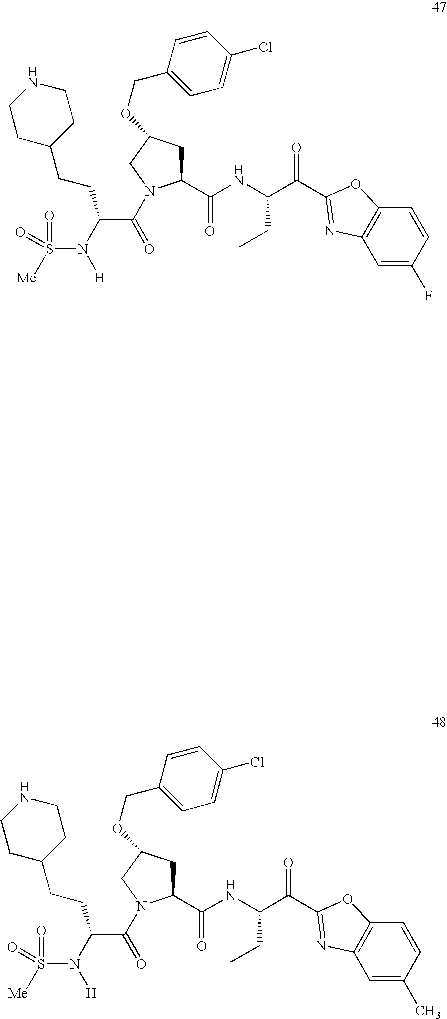

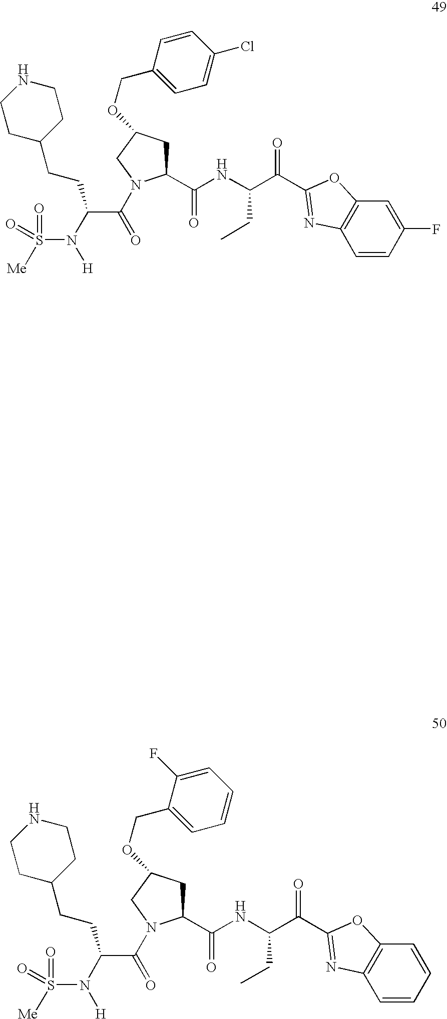

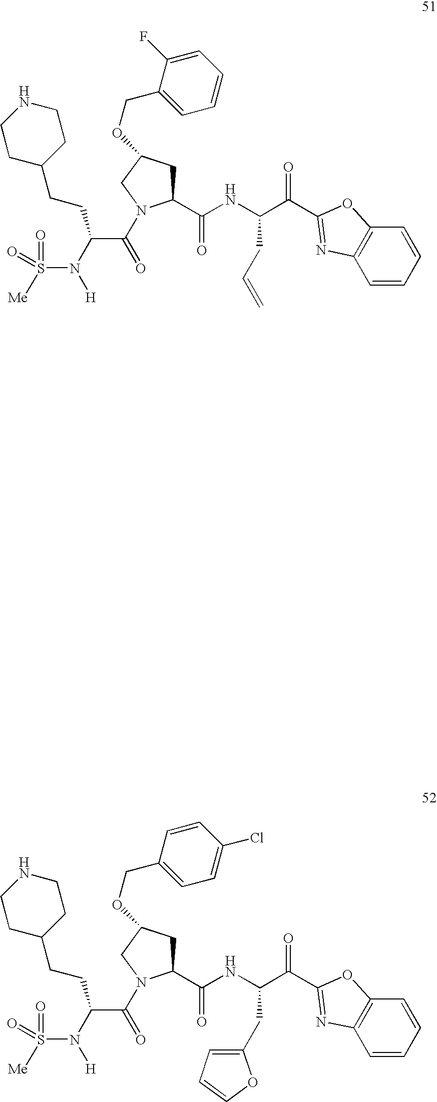

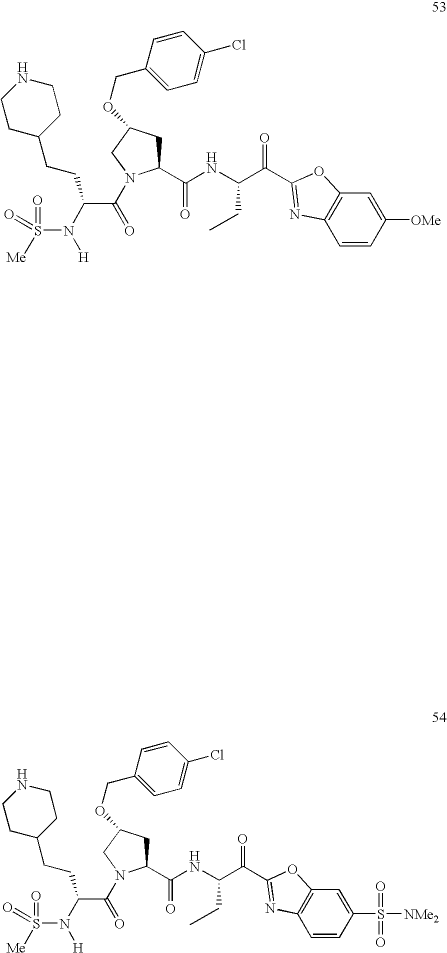

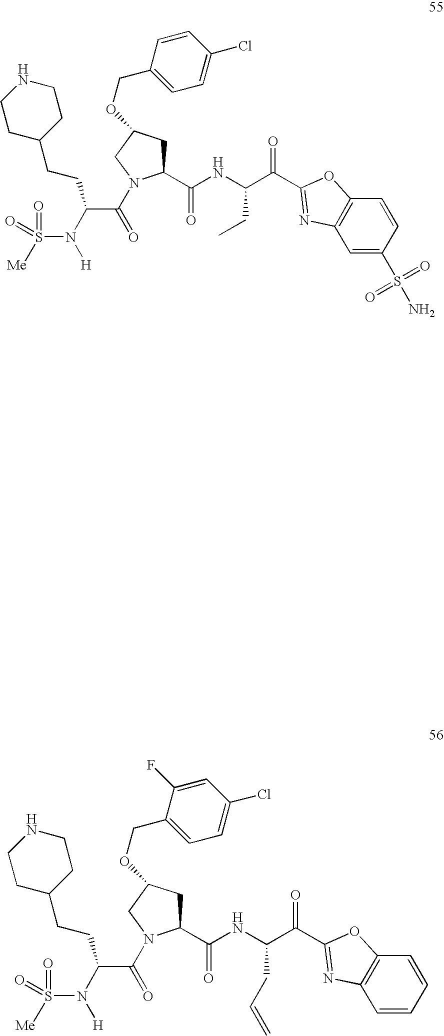

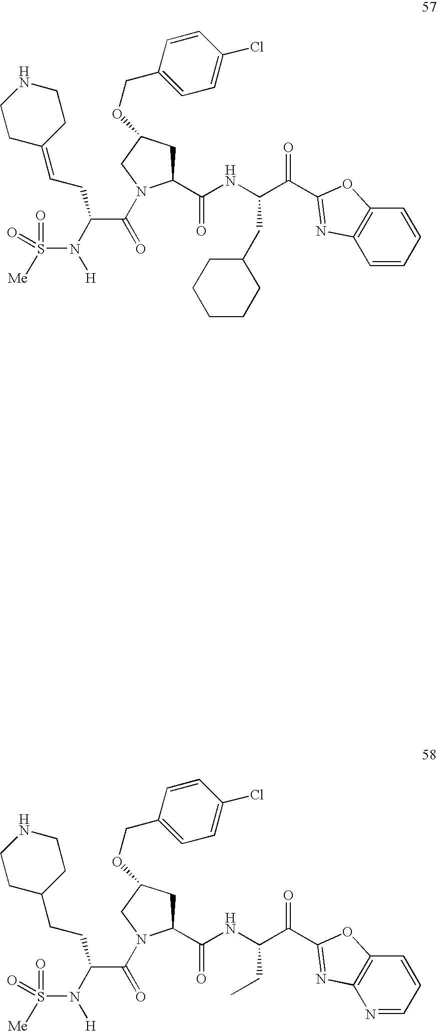

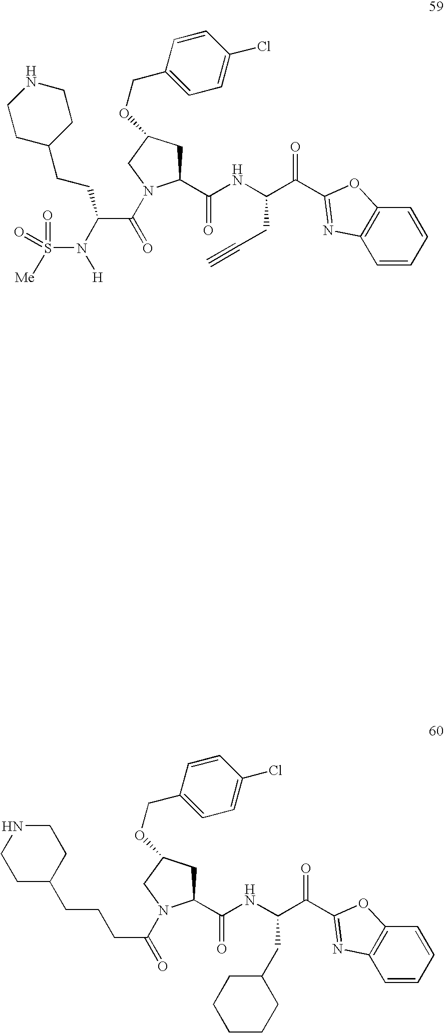

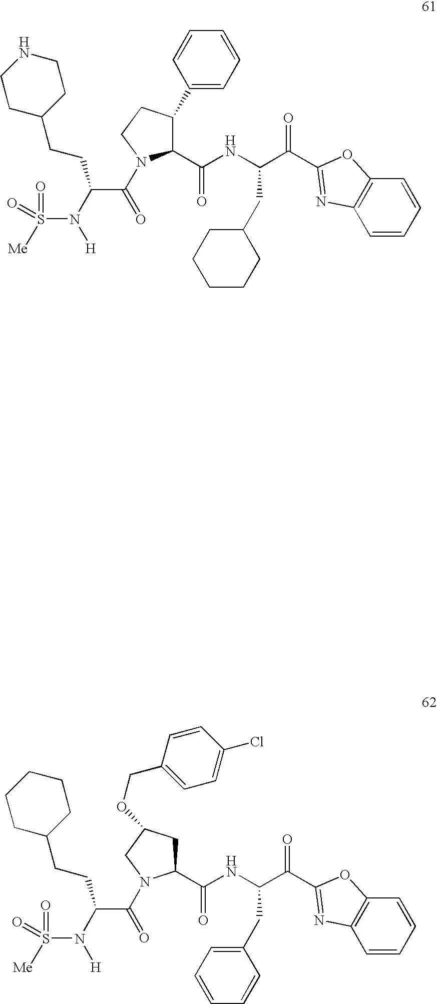

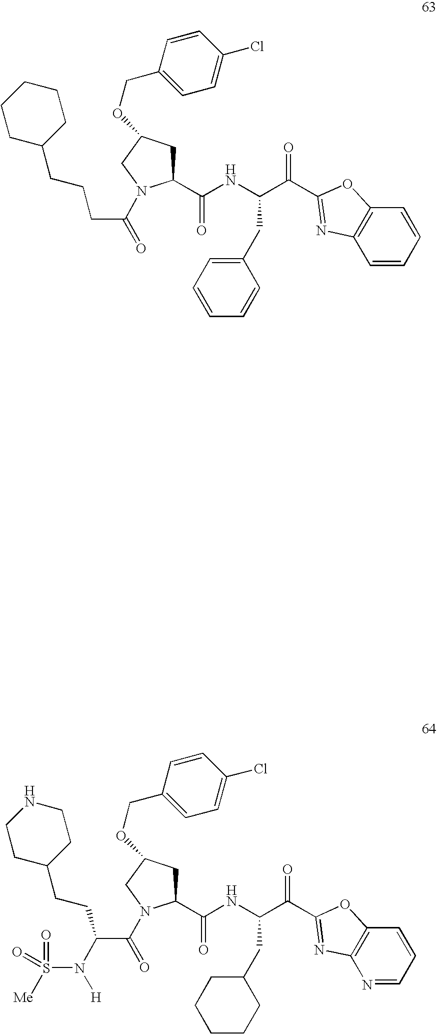

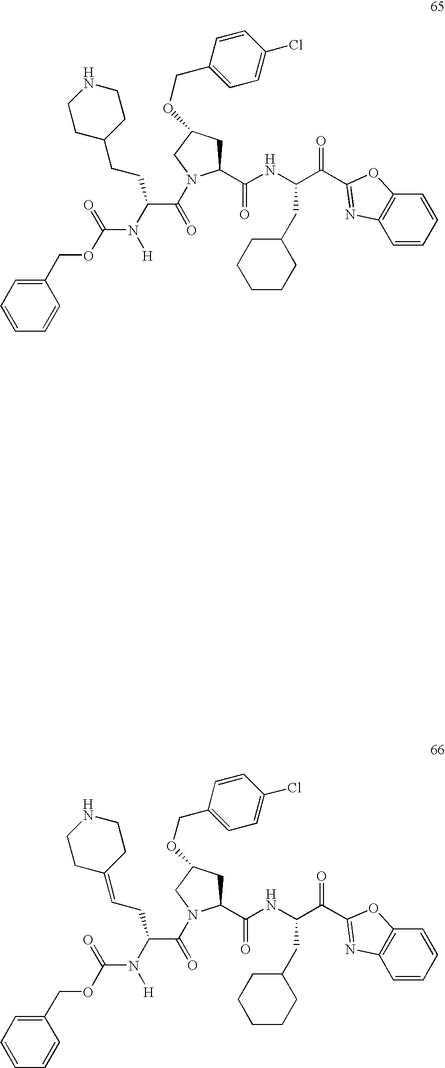

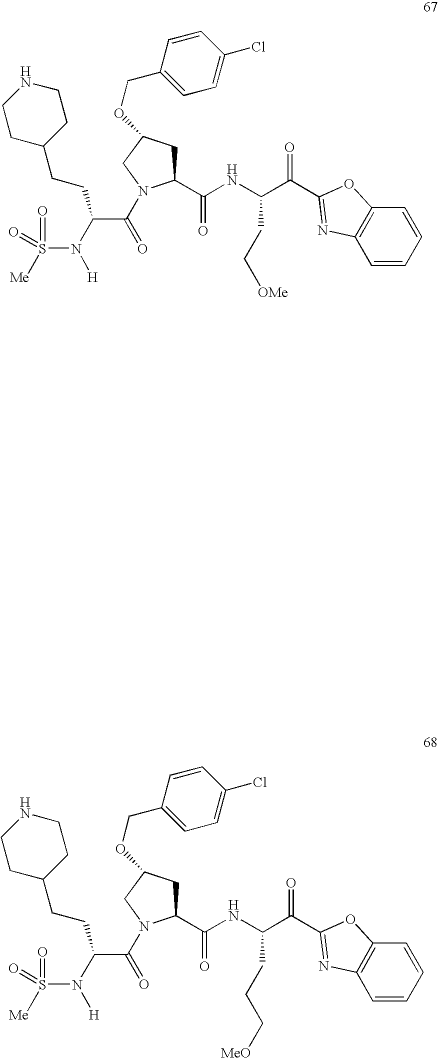

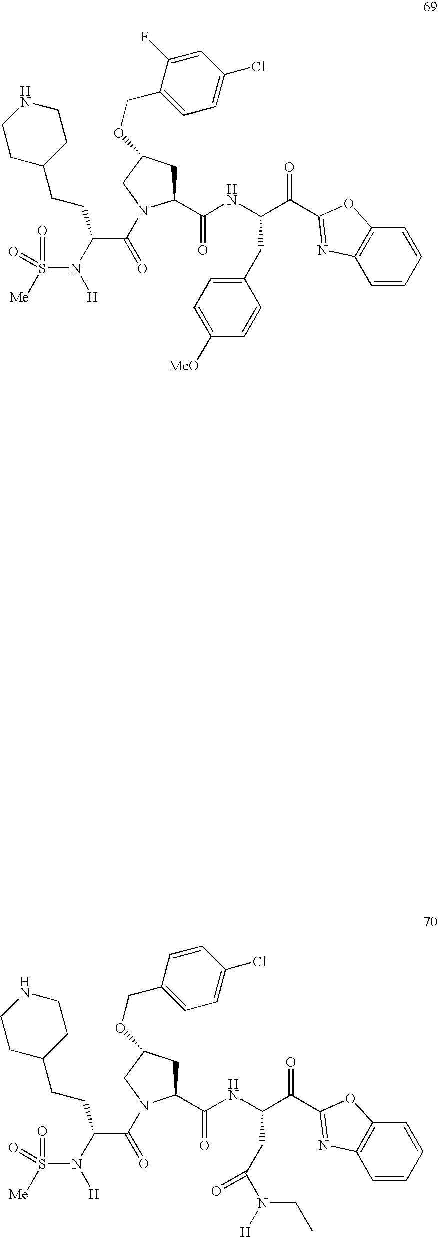

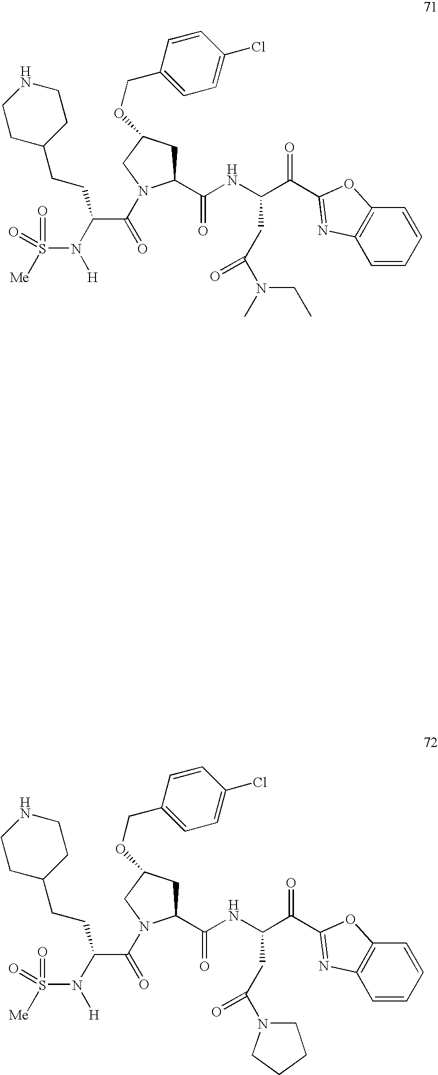

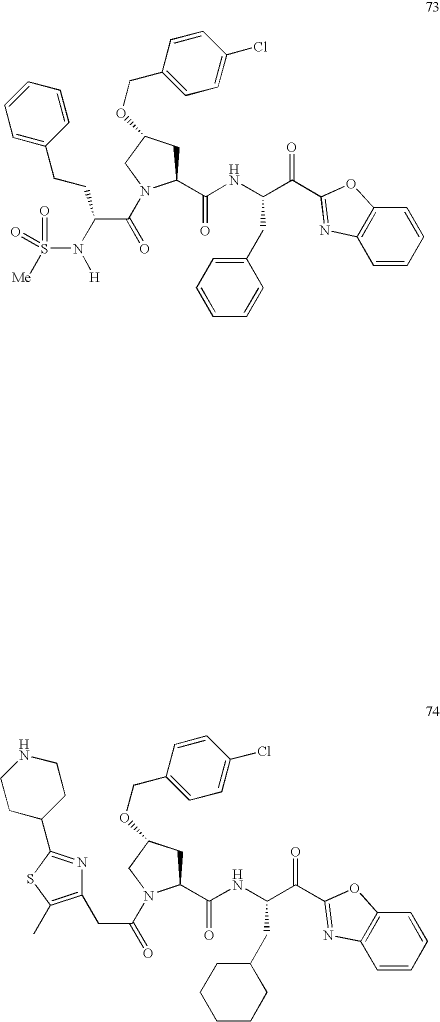

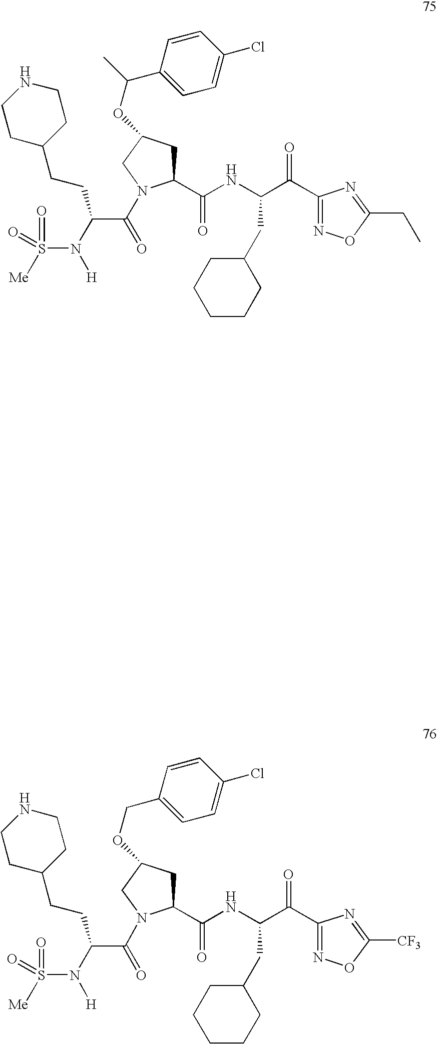

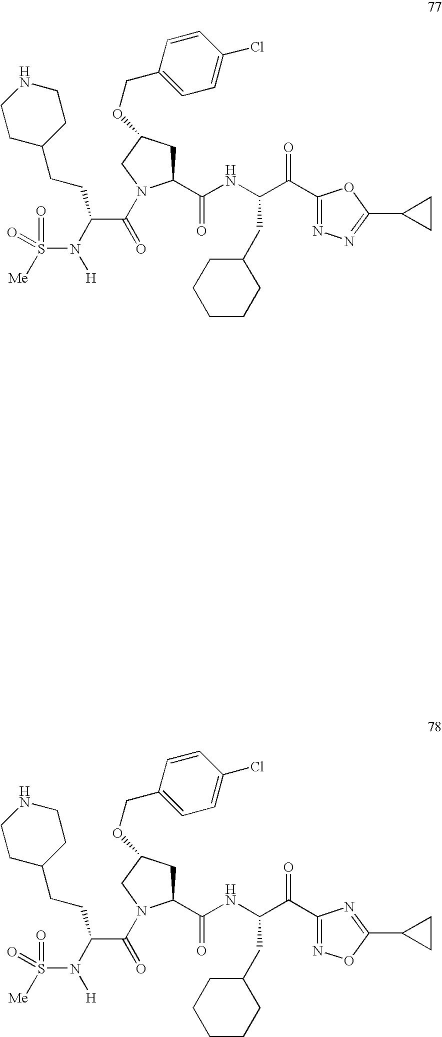

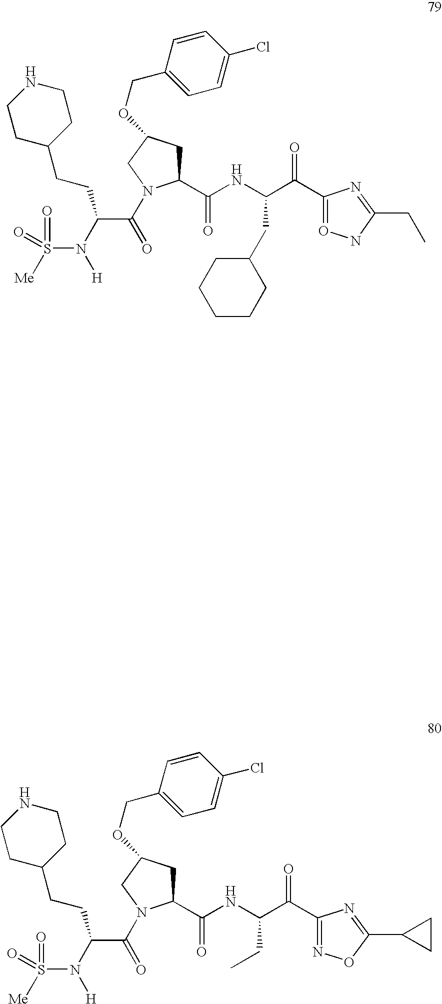

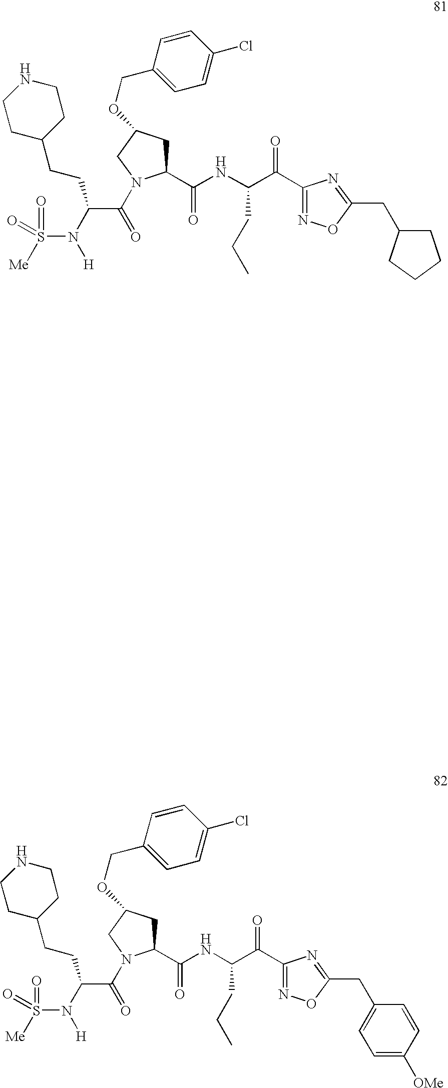

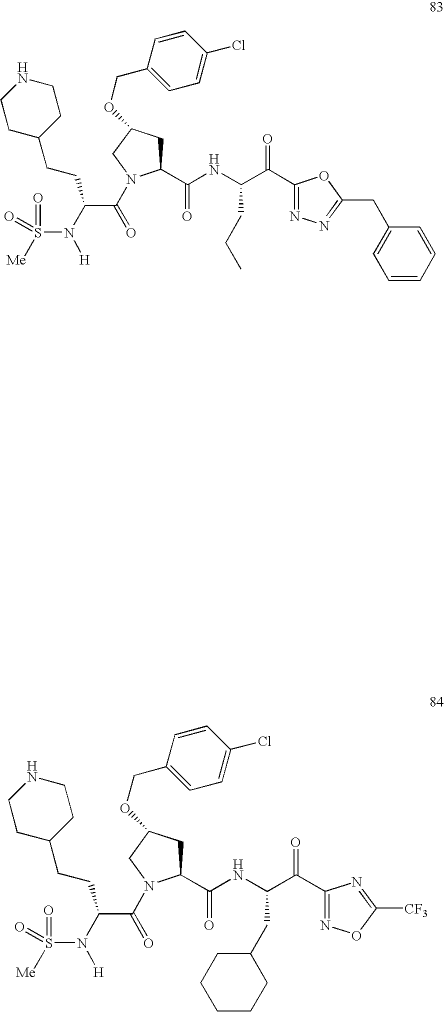

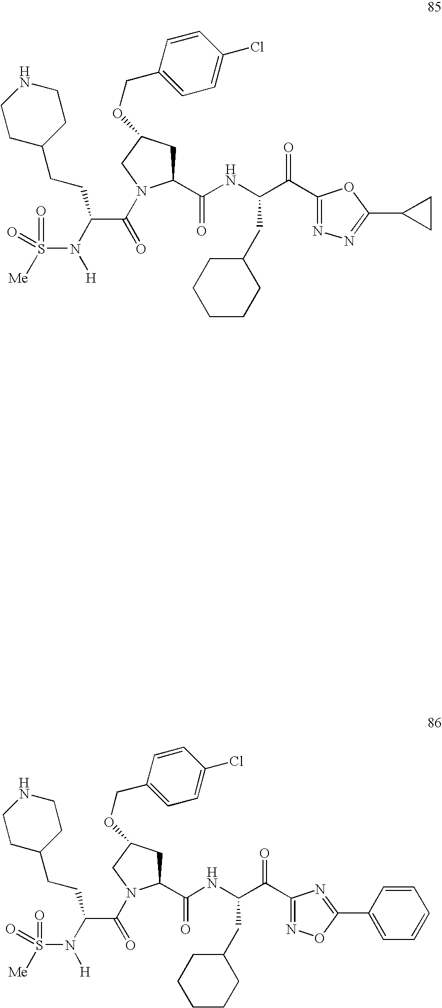

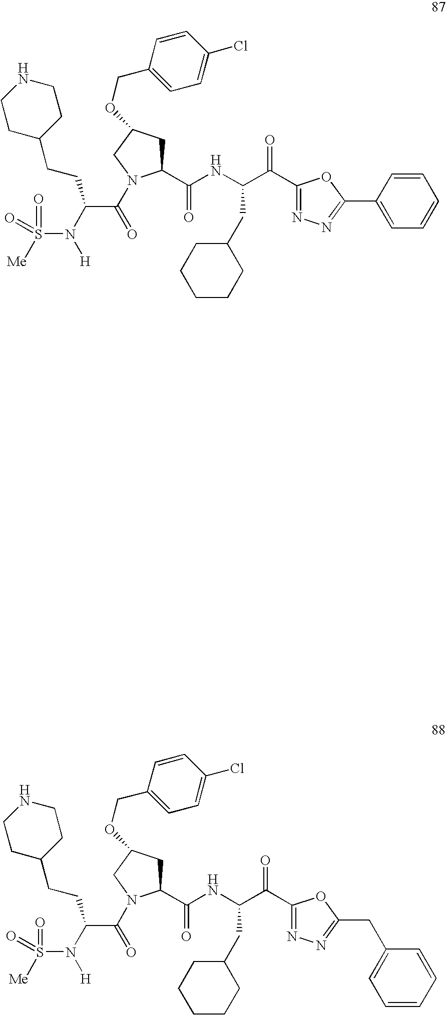

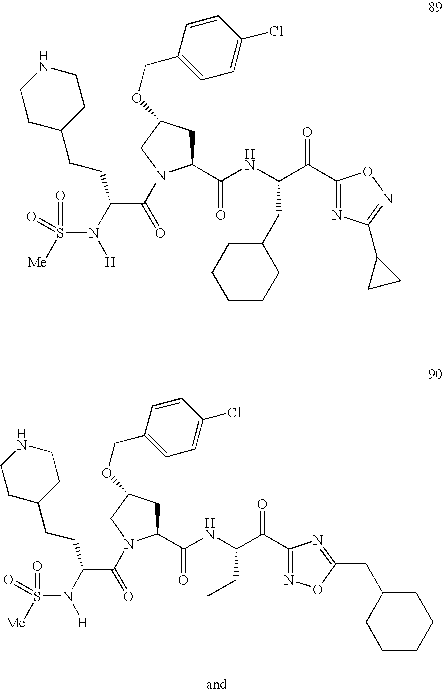

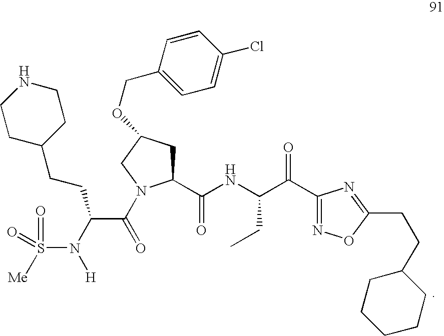

15. A pharmaceutical composition comprising a therapeutically effective amount of a compound of claim 1.

16-22. (canceled)

23. A method for inhibiting a channel activating protease, comprising contacting a cell or tissue system or a mammal in need of treatment thereof, with a compound of claim 1, wherein said channel activating protease is prostasin, PRSS22, TMPRSS11 (e.g., TMPRSS11B, TMPRSS11E), TMPRSS2, TMPRSS3, TMPRSS4 (MTSP-2), matriptase (MTSP-1), CAP2, CAP3, trypsin, cathepsin A, or neutrophil elastase.

24. A method for treating a condition mediated by a channel activating protease, comprising contacting a cell or tissue system or a mammal in need of treatment thereof, with a compound of claim 1 and optionally in combination with a second therapeutic agent; wherein said channel activating protease is prostasin, PRSS22, TMPRSS11 (e.g., TMPRSS11B, TMPRSS11E), TMPRSS2, TMPRSS3, TMPRSS4 (MTSP-2), matriptase (MTSP-1), CAP2, CAP3, trypsin, cathepsin A, or neutrophil elastase.

25. The method of claim 24, wherein said condition is associated with the movement of fluid across ion transporting epithelia or the accumulation of mucus and sputum in respiratory tissues, or a combination thereof.

26. The method of claim 24, wherein said condition is cystic fibrosis, primary ciliary dyskinesia, lung carcinoma, chronic bronchitis, chronic obstructive pulmonary disease, asthma or a respiratory tract infection.

27. The method of claim 24, wherein said second therapeutic agent is an anti-inflammatory, bronchodilatory, antihistamine, anti-tussive, antibiotic or DNase, and is administered prior to, simultaneously with, or after the compound of claim 1.

28. The method of claim 24, wherein said channel activating protease is prostasin.

29. The method of claim 24, wherein said cell or tissue system comprises bronchial epithelial cells.
